SEQUENCE LISTING (98 sequences)

SEQ ID NO: 1 - DNA, 342 bp, Homo sapiens
  CDS (1)..(342)
gaaattgtgttgacgcagtctccaggcaccctgtctttgtcaccaggggaaagagccaccctctcctgcaggtccagccagagtgttttatacagctccaacaataagaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaacgt

SEQ ID NO: 2 - protein, 114 aa, Homo sapiens
EIVLTQSPGTLSLSPGERATLSCRSSQSVLYSSNNKNYLAWYQQKPGQAPRLLIYGASSRATGIPDRFSGSGSGTDFTLTISRLEPEDFAVYYCQQYGSSPWTFGQGTKVEIKR

SEQ ID NO: 3 - DNA, 360 bp, Equus caballus
caggtgcaactgaaggagtcaggacctggcctggtgaagccctcgcagaccctctccctcacctgcactgtctctggattatcagtgagcagtaatggtgtggcctgggtccgccaggctccaggaaaagggctggaatttgtcggtgttatacatactgatggaggtgttgactacaacccagccctgaagtcccgaggcagcatcactagggacatctcaaagagccaactttatctgacgctgaacacactgacaggcgaggacacggccgtctattactgtgcgcgacatgctagtactggtgcttacctttacccctttgactattggggccagggtatcctggtcaccgtctcg

SEQ ID NO: 4 - DNA, 342 bp, Equus caballus
caggtgcaactgaaggagtcgggacctggcctggtgaagccctcgcagaccctctccctcacctgcactgtctctggattctctttgaacacttacgcagtgggatgggtccgccaggctccaggaaaaggcctggaatttgttggtagtatttatagtattggaagtgcgacgtacaatttagacctgaagtcccgagtcagcatcaccaaggacacctcaaagagccaagtttatctgacggtgaatagtctgacaagtgaggacacggccgtctattattgtggaagacgagtcaatgaaattgactactggggccagggtatcctggtcaccgtctcg

SEQ ID NO: 5 - DNA, 369 bp, Equus caballus
caggtgcaactgaaggagtcaggacctggcctggtgaagccctcgcagaccctctccctcacctgcactgtctctgggtcatcttcggagggttatggtgtgggctgggtccgccaggctccaggacgaggactagagtttgtagggggtataaccaatagtggtagtgcaagatttaatccaggactggcgtcccgagccagcattctcaagaacaccgaaaagagccaagtttacctgacgctgaccgacctgacaggcgaggacacggccgtctattattgtgcgaaggattccgagagtggctttctttattggggacattacggtgtagaatattggggccagggtatcctggtcaccgtctcg

SEQ ID NO: 6 - DNA, 375 bp, Equus caballus
caggtgcagctgcaggagtcaggacctggcctggtgaagccctcgcagaccctctccctcacctgcactgtctctgggttatctttgagcagtaatactgtaggctgggtccgccaggctccaggaaaaggactggaatacgttggtgctatatatggtagtgcaagtgcagcgtacaacccagccctgaagtcccgagccagcatcaccaaggacacctcaaagagccaagtttatctgacgctgaacagcctgacaagcgaggacacggccgtctattactgtgcaggaggaggcggtggttggattggttatgactacttaggatattatgatataaactactggggccagggtatcctggtcaccgtctcg

SEQ ID NO: 7 - DNA, 351 bp, Equus caballus
caggtgcagctgaaggagtcgggacctggcctagtgaagccctcgcagaccctctccctcacctgcactgtctctggattatctgacaacagtaacgctgtgggctgggtccgccaggctccaggaaaaggactggaatttgtggctgatctaacggatagtgacagtaacccagccctgaagtcgcgagtcaggatcaccaaggaaccctcaaagagccaagttcgcctgattatgaacagcctgacagaagaggacacggccgtctattactgtattcatggttactacaatagttttatggtgggagcgataaaatattggggccagggtatcctggtcaccgtctcg

SEQ ID NO: 8 - DNA, 378 bp, Equus caballus
caggtgcaactgaaggagtcaggacctggcctggtgaagtcctcgcagaccctctccctcacctgtactgtctctggggcgtccttgaacgacattgctgtgggttgggtccgccaggctccaggaaaaggactggaatacgttggttgtgtttatgatggtaccggagaaaactataacccagccctgaagtcccgagccagcatcaccagggacacctcaaagagccaggtttatctggcgctgaacagcttgacgagtgaggacacggccgtctattattgtacaggaggcaagggtgactatggtagatactggaatagttacgctgaggatggaataaccaactggggccagggtatcctggtcaccgtctcg

SEQ ID NO: 9 - DNA, 378 bp, Equus caballus
caggtgcaactgaaggagtcaggacctggcctggtgaagccctcgcagaccctgtccctcacttgcactgtctctggattctctttgatcactgacagtgtaggctgggtccgccaggctccagggaaagggctggaatttgttggtggactttctagttttggaagtgcaaattacaacccaggcctgaactcccgagccagcatcaccaaggacacctcaaagggccaagtcgttctgacgctgaacagcctgacaagcgacgacacggccgtctattactgtgtgtcattttcgggccagggtgaagcgttcgctttcgcttacctttattatggaataacctactggggccagggtatcctggtcaccgtctcg

SEQ ID NO: 10 - DNA, 345 bp, Equus caballus
caggtgcaactgcaggagtcaggacctggcctggtgatgccctcgcagaccctctccctcacctgcactgtctctggattatctctggtgaacaggaatgctgtgcgctgggtccgccaggctccgggaaaagggctggaatacgttggttcaatatacggtgttgaagaacgaaactacaacccagtcctgaagtcccgagtcgatatcaccaaggacacctcaaagagtcaagtttatctgacgctgaatagcgtgacaagcggggacacggccgtctattactgtgcgagaaatgaatatggtattgtggaatggggccagggtatcctggtcaccgtctcg

SEQ ID NO: 11 - DNA, 363 bp, Equus caballus
caggtgcagctgaaggagtcaggacctggcctggtgaagccctcgcagaccctctccctcacctgcactgtctctggattatctttgagcagtaatactgtagggtgggtccgccaggctccaggaaaagggctggagtacgtcggtattatctatggtagtgcaagtacattgtacaacccagccctgaagtcccgagccagcatcaccaaggaatcctcaaagagccaagtttatttgacgctgaacagcctgacaagcgaggacacggccgtctattattgtgcaggaggctttagcggctttgattggttcgatagaggtataaactactggggccagggtatcctggtcaccgtctcg

SEQ ID NO: 12 - DNA, 369 bp, Equus caballus
caggtgcaactgaaggagtcgggacctggcctggtgaagccctcgcagaccctcagcctgacatgcagtgtctctggattgaatttgaacgaagatattgtagggtgggtccgccaggctccaggaaaagggccggaatacgtcggaagtatatggggagatagaagcccaaaatacaatccagacgtgaagtcccgagccagtatcagtaaggacacctcgaaacgccaggtttatcttcaactgaacagcctgagtgacgaggacacggccgtctattactgtgcaggaggacttacaattttaggcgtcatgaaggatgagacgttcgtggatcactggggcccgggtatcctggtcaccgtctcg

SEQ ID NO: 13 - DNA, 363 bp, Equus caballus
caggtgcagctgaaggagtcaggacctggcctggtgaagccctcgcagaccctctccctcacctgcactatctctggattatctttgagcagctatggtgtgggctgggtccgccaggctccaggaaaagggctggaattcgttggtggaatacgtagtagtggaagtgcaaactacaatccagccctgaagtcccgagccagcatcaccaaggacacctcacagagccatgtttatctgacgctgaacagcctgacaagcgaggacacggccgtctattactgtgcaggagggacagaacaacgtgattatattgacgttggtgtgaagttctggggccagggtatcctggtcaccgtctcg

SEQ ID NO: 14 - DNA, 339 bp, Equus caballus
caggtgcaactgaaggagtcaggacctggcctggtgaagccctcgcagaccctctccctcacctgcactgtctctggattatctttgagcagtgttgatgtaggctgggtccgccaggctccaggaaaaggactggaatacgttagttggataggtagaagtactagctacaagccggccctgaagtcccgagccagcatcaccaaggacacctcaaagagccaagcttatctgacgctgaacagtctgacgagcgaggacacggccgtctattactgtgtaggaggttacgcggacggtatagattactggggccagggtatcctggtcaccgtctcg

SEQ ID NO: 15 - DNA, 363 bp, Equus caballus
caggtgcagctgaaggagtcaggacctggcctggtgaagatctcgcagaccctctccctcacctgcactgtgtctggattatctttgagcagtaatgatgtaggctgggtccgccaggctccaggaaaagggctggaatacgtggctcgtatatggggtggtgcaaatgaacactacaacccagccctgaagtcgcgagccagcatcaccaaggacacctcaaagagccaagtttatctgacgctgaacagcctgacaagcgaggacacggccgtctattactgtggaggaacacctggtttctataatagtgcttacgagacgtttgcctactggggccagggtatcctggtcaccgtctcg

SEQ ID NO: 16 - DNA, 357 bp, Equus caballus
caggtgcaactgaaggagtcaggacctggcctggtgaagccctcgcagaccctttccctcacctgcactgtctctggattacttttgaacagtaattgtgtaggttgggtccgccaggctccaggaaaacgactggaatacgttggttctatatatgggacgttaacaaactacaactcagccctgaggtcccgagccagaatcaccagcgactactcaaagagccaagttcttctgacgctgaacagcctgacaagcgaggatacggccgtctattactgtgcagcactcgattatggtgtgacgattagtcgcgatataaatgattggggccagggtatcctggtcaccgtctcg

SEQ ID NO: 17 - DNA, 342 bp, Equus caballus
caggtgcagctgaaggagtcaggacctggcctggtgaagccctcgcagaccctctccctcacctgcactgtctctggattgtctttgacaggtagtcagatagcttgggtccgccaggctccaggaaaaggactggaatatattagtggaagttcaatgtacaacccagccctgaagttccgagccagcatcaccaaggacacctccaagaatcaagttactctgacgctgaataagctgacaggcgaggacacggccgtctattactgtgtggcgacagctttttggggcggttatggcggtatccaatactggggccagggtatcctggtcaccgtctcg

SEQ ID NO: 18 - DNA, 366 bp, Equus caballus
caggtgcagctgaaggagtcaggacctggcctggtgaagccctcgcagaccctctccctcacctgcactgtctctggattatctttgacagattatggtgtgggctgggtccgccaggctccaggaaaagggctggaatttgttgccagaatagatagtgatggaagtaaaaactttaacccagcgctgaagtcccgagccaacatcatcaaggacacctcaaagagccaagtttatctgacgctgaacagcctgacaagtgaagacacggccgtctattactgtgcagggtatggttacagtggtcgttactccacaccggggaatttatactggtggggccagggtatcctggtcaccgtctcg

SEQ ID NO: 19 - DNA, 354 bp, Equus caballus
caggtgcagctgaaggagtcgggacctggcctagtgaagccctcgcagaccctgtccctcgtctgcactgtcagtggattctccctgaccgaccggggtgtaggctgggtccgccaggcgccaggaaaaggactggaatttgtgagttatatactaaccagtggagcccaagacgggaatccagccctaaggtcccgagtcagcatcaccagggacacctcactgagtcaagtttatctgacaatgaacagcgtgacaggcgaggacacggccgtctactattgtgggaggcatggaccgaatcttcatggaacttttgactattggggccagggtatcctggtcaccgtctcg

SEQ ID NO: 20 - DNA, 369 bp, Equus caballus
caggtgcagctgaaggagtcaggacctgacctgatgaagccctcgcagaccctctccctcacctgcactgtctctggattctctttgagcagttatggtgtaggctgggtccgccaggctccaggtaaaggcctggagtttgttggcgggttacctggtagtggaagtgcagactacagcccagccctgaggtcccgagccagcatcaccaaggacacctcaaagagccaagtttatgtgacgctgaacagcctgacaagcgaggacacggccgtctattactgtgcaagattctataactggaatagtggtgttgtcagttatactggtattgactactggggccagggtatcctggtcaccgtctcg

SEQ ID NO: 21 - DNA, 372 bp, Equus caballus
caggtgcaactgaaggagtcaggacctggcctggtgaagccctcgcagaccctctccctcacctgcactgtctctggattatctttgagcagttatggtgtggactgggtccgccaggctccaggaaaaggacttgaatgggttggtggtataactagtagtggaggttcaggttacaacccagccctgaagtcccgagccagcatcaccaaggacacctcaaagagccaagtttatctgacgctgaacagcctgacaagcgaggacacggccgtctattactgtgcaggagaggaggaaggctacgtttatggttttactcgttattatggtaactactactggggccagggtatcctggtcaccgtctcg

SEQ ID NO: 22 - DNA, 363 bp, Equus caballus
caggtgcagctgcaggagtcaggacctggcctggtgaagccctcgcagaccctctccctcacctgcagtgtctctggattgtctttgagcagtgtttttgtatactgggtccgccaggctccaggaaaagggctggaatatgttggttttataggtaatagtggaagtacaataggtaatagtggaaaaacaaactacaactacaacccagtcctgaagtcccgagccagcatcagcaaggacacctcaaagagccaagttcttctgacgctgaacagcctgacaagcgaggacacggccgtctattactgtgcaggagacaatataaagtattggggccagggtatcctggtcaccgtctcg

SEQ ID NO: 23 - DNA, 369 bp, Equus caballus
caggtgcaactgaaggagtcaggacctggcctggtgaagccctcgcagaccctctccctcatctgcactgtctctggattctctttgagcagtgacagtgtaggctgggtccgccaggctccaggaaaagggctggaatttgttggagtggtacatagtagtggaagggcaagaaacccagccctgaagtcccgagccagcatcaccaaggacacctcagagagccaagtttatctgacgctgaacagcctgacaagcgaggacacggccgtctattactgtgcaggggggcgtagtggctacagttattacgctgggatggtagatggtataaactactggggccagggtatcctggtcaccgtctcg

SEQ ID NO: 24 - DNA, 366 bp, Equus caballus
caggtgcaactgaaggagtccggacctgacctggtgaagccctcggagaccctctccctcgtctgctccgtctctggacaatctttgagcagttatgatgtgggctgggttcgccaggctccaggctggggactggaattcgttggtgtaacggcgcattatggaggtatagactacaatccagccctgaagtcccgagccagcatcaccaaggacacctcaaagaaccaacttactctgatactgaatagtctgacaagcgaggacacggccgtctattactgtacaggagaagcgcagactaattgtgactttggcgtcagttgtttgggctactggggccagggtatcctggtcaccgtctcg

SEQ ID NO: 25 - DNA, 357 bp, Equus caballus
caggtgcaactgaaggagtcgggaccgggcctggtgaagccctcgcagaccctctccctcacctgcactgtctctggattaagtttgagcagttatggtgcaggctgggtccgccagtctccaggaaaagggctggaatatgttggtggggtgggtaaaagtggaagttcaaattacaattcagccctgaagccccgagccagtatcaccaaggactcctcaaagagtcagatttctctgacgctgagaagcctgacaggcgaggacacggccgtctattactgtgcgatctacgatagttatcttcgtggttggtcagttgtctactggggccagggtatcctggtcaccgtctcg

SEQ ID NO: 26 - DNA, 363 bp, Equus caballus
caggtgcagctgaaggagtcaggacctggcctggtgaagccctcgcagaccctctccctcacctgcactgtctctggattatctttgagaggtaatgttgtaggctgggtccgccaggctccaggaaaagggctggaacacgttggcgaaaacgttagtagtggaggtgcgttctacagcccagccctaaagtcccgagccagcatcaccagggacacctcaaagagccaaatttatctgacgctgaacagcctgacaagggaggacacggccgtctattactgtgcagcatggaaggttagcagtcgctcttacttggatggtataaactactggggccagggtatcctggtcaccgtctcg

SEQ ID NO: 27 - DNA, 336 bp, Equus caballus
caggtgcaactgaaggagtcaggacctggcctggtgaagccctcgcagaccctctccctcacctgcgctgtctctggattctctttgagcagtgacggtataaactgggtccgccaggctccaggaaaagggctggaattcgtgggttctatatatactagtgcaagtacaatctacaacccagccctgaagtcccgagccagcatcaccaaggacacctcaaagagccaagtttatctgacgctgaacagcctgacaagtgaggacacggccgtctattactgttcaggaggcagtgaagaatattggggccagggtatcctggtcaccgtctcg

SEQ ID NO: 28 - DNA, 372 bp, Equus caballus
caggtgcaactgcaggagtcaggtcctggcctggtgaggccagcagagaccctctccctcacctgcactgtctctggattggacttgagcagtggtacgataatctgggtccgccaggctccaggaaaagggctggagagagtcggtgaaatagttggtgagggaagtggattctacaatccagccctgaagtcccgagccatgatcaccaaggacacctcgaagaatgagatttatctgacactgaagagcctgacaagcgaggacacggccgtctattactgtgcaggagcctggggcggaaattactacgaaaatttttttattaatggtgtagagaattggggccagggtatcctggtcaccgtctcg

SEQ ID NO: 29 - DNA, 372 bp, Equus caballus
caggtgcagctgaaggagtcgggacctggcctggtgaagccctcgcagaccctctccctcacctgcactgtctctggattatctttgagcagtagttgtgtacaatgggtccgccaggttccaggaaaagggctggaatacgtcggtaggatagttagtagtggtggtggtctaacctacaacccggccctgaagtcccgagccagcatcaccagagacacttcaaagagccaggtttatctgacgctgaacagcctgacagacgaggacacggccgtctattactgtacaggggccctgaatactcactacagttcatacgcgggttatggtatagactactggggccagggtatcctggtcaccgtctcg

SEQ ID NO: 30 - DNA, 372 bp, Equus caballus
caggtgcagctgaaggagtcagggcctggcctggtgaagcccgcgcagacccttacccttacctgcactgtctctggattacacttgaacagtgacgcggtagtgggctgggtccgtcaggctccaggaaaggggctggaatttgttggtggattgtctaatacaggacgtgcaaactacaatccagccctgaagtcccgagccatcatcaccaaggacacctcaaagagccaggtttatctgaccctgaacagcctgacaagcgaggacacggccgactatttttgtgcaggaggtagaatgttcgattatgtttatggcggctattacgaaatacaatattggggccagggtatcctggtcaccgtctcg

SEQ ID NO: 31 - DNA, 387 bp, Equus caballus
caggtgcaactgaaggagtcgggacctggcctggtgaagccctcgcaggccctgtccctcacctgtactatctctgggttctctttgaccagtcacggtgtaggctgggtccgccaggctccaggaaaagggctggaatttgtcggtagtatatggactacgggacagacaatcaacaatccaaccctgaagtcccgagtcagcatcactagggacaccgggctgaaccaagtttcactgacgttgaatgagttgacaagcgaggacacggccgtctattattgtgcaggaggcgcgatttccgattacgactttttcggttttcgtggtatgttcagcatttatgatgtgcagtattggggccagggtatcctggtcaccgtctcg

SEQ ID NO: 32 - DNA, 366 bp, Equus caballus
caggtgcagctgaaggagtcaggacctggcctggtgaagccctcgcagaccctctccctcgtctgcactgtctctggattcagtttgaacagttggggtgtaggctgggtccgccaggctccaggaaaagggctggaggaagttggtggaagtcagattggtgggaatgcaaactacaatccagccctggagtcccgagccagcatcaccaaggacgcctcaaagagccaagtttatctgacgctgaacagcctgacagaagaggacacggccgtctattactgtacaggaggttacaactggaatcttgggactaatagggaccgtataacgtactggggccagggtatcctggtcaccgtctcg

SEQ ID NO: 33 - DNA, 381 bp, Equus caballus
caggtgcagctgaaggagtcaggacctggcctggtgaagcccgggcagaccctctccctctcctgcactgtctctggattgtcattgagcacaaatactgtaggttgggtccgccaggctccaggaaaaggatgggaatatgttgctgcgttatacgcggatgcagatggagattataatccagtccttcagtcccgagccagcatcacgaaggacacctccaagaaccaggtctttctgacgctagacacactgacgagcgaggacacggccgtctattactgcacagggggagtcttctccgtccccgtcggtactggatatacttactatgaatcgggaatactatactggggccagggtatcctggtcaccgtctcg

SEQ ID NO: 34 - DNA, 363 bp, Equus caballus
caggtgcaactgaaggagtcaggacctggcctggtgaacccctcgcagaccctgtccctcacctgctttgtctctggattctctttgacgagttggcatgtaggctgggtccgccaggctccaggaaaagggctggaatttgtcggtggtatacctgtcatcggagaggcatactacaacccagtgctgaagtcccgaatcagcattactaaggacacctcgaagagccaagtttatctgacgctgaacagcctgacagacgaggacacggccgtctatgcctgtgcgaggttaaggaactggtatggtgattactacagtgacatggactattggggccagggtatcctggtcaccgtctcg

SEQ ID NO: 35 - DNA, 342 bp, Equus caballus
caggtgcagctgcaggagtcaggacctggcctggtgaagccctcgcagaccctgtccctcacctgcactgtctctggactctctttgaacagttacgatgtaaactgggtccgccaggctccaggaaaagggctggaagtagttggtagtataagtgacagtggaattgcggtgtacaacccagccctgaagtcccgagccagcatcaccaaggacacctcaaacagtcaagtttatctgacgctgaacagcctgacaggcgaggacacggccgtctattactgtgcgagagggaattttgcgtttgactactggggccagggtatcctggtcaccgtctcg

SEQ ID NO: 36 - DNA, 336 bp, Equus caballus
caggtgcaactgaaggagtcaggacctggcctggtgaagccctcgcagactctctccctcacctgcgctgtctctggattctctttgagagatgccgccatgggctgggtccgccaggctccaggaaggggtctggaatacatcggttctatgtatattagagaagactacaatccagccctgaagtcccgagccagcgtcaccaaggacacaaaggagagccgaagttatctgacactgaacgcgctgacaagtgaggacacggccgtctattggtgtgtaggggatgttggcactggatactactggggccagggtatcctggtcaccgtctcg

SEQ ID NO: 37 - DNA, 357 bp, Equus caballus
caggtgcagctgaaggagtcgggacctggcctggtgaagccctcgcagaccctctccctcacctgcactgtctctggattctctttgagcaccaccggtgtgggctgggtccgccaggctccaggaaaagggctggaatttgttggtggtgtacctagtagtggaagtgcaaactacaatccagccctgaagtcccgatgcagcatcaccaaggacgaatcaaagagccaagtttatctgacgctgaacagcctgacaagcgaggacacggccgtctatatatgtgcaggaggcttctacaatacattggataaggggataaactattggggccagggtatcctggtcaccgtctcg

SEQ ID NO: 38 - DNA, 357 bp, Equus caballus
caggtgcaactgaaggagtcaggacctggcctggtgaagccctcgcagaccctgtccctcacctgcactatctctggattctctttgaccagtgccagtgtagactgggtccgccaggctccaggaaaagggctggaatttgttggtggtatagcgactagtgggcgtgcaaattacaacccagtcctgaagtcccgcgccactatcaccagagacacctcaaagagccaagtttatctgacgctgaacagtctgacaggcgaggacacggccgtctattactgtgcggaatcctactatgatggtgttggtggtaattactacttttggggccagggtatcctggtcaccgtctcg

SEQ ID NO: 39 - DNA, 378 bp, Equus caballus
caggtgcaactgaaggagtcaggacctggcctggtgaagccctcgcagaccctttccctcacctgcaccgtctctggaatgtctttgagcaccaacactgtaggctgggtccgccaggctccaggaaaagggctggaatacgttggtctaatctatggtatgaaaagtgcagagtacaatccagccctgaagtcccgagccagtatcaccaaggacacctcaaatagtcaagttcttctgacgctgaatagcctgacaagcgaggacacggccgtctactactgtgcagggggtgaagcctggggtccaatgtatagttcgaacgaagaaaaaaatggtgtggaatactggggccagggtatcctggtcaccgtctcg

SEQ ID NO: 40 - DNA, 351 bp, Equus caballus
caggtgcaactgcaggagtcaggacctggcctggtgaagccctcgcagaccctctccctcacctgcactgtctctggaatctctttgaccgattacaatgtagactgggtccgccaggctccaggaaaagggctggaatttgttggtggactatggactaatggacaatcgaactacaatccagccctgaagtcccgagccagaatcaccaaggacacctcaaagagtcaagtttatctgacgctgaacagcctgacaagcgaggacacggccgtctattactgtgagggttatggtaattcctggcagccaccgcactactggggccagggtatcctggtcaccgtctcg

SEQ ID NO: 41 - DNA, 348 bp, Equus caballus
caggtgcagctgaaggagtcaggacctggcctggtgaagccctcgcagaccctgtccctcacctgcactgtctctgggaacgatttgagaagttttggcgtagcctgggtccgccaggctccaggaaaaggcctggaatttgttggtggtgtagccaggtttggcagcccttactacaacccagccctgaagtcccgggccatcatcaccaaggacacctcaaagaaggaaagtgtgctgacgttaaatagcgtgacaggcgaggacacggccgtctattggtgtgcagggggatatggtgatgaatcctggggaccctggggccagggtatcctggtcaccgtctcg

SEQ ID NO: 42 - DNA, 369 bp, Equus caballus
caggtgcaactgaaggagtcaggacctggcctggtgaagccctcgcagaccctctccctcacctgcactgtctctgcgttatctttgagcagtgctggtgtgggctgggtccgccaggctccaggaaaagggctggaatttgttgctggtatagttggtgatggtggtacgtacgccaacccagccctgaggtcccgagccagcatcaccaaggacacctcaaagagccaagtttatctgacgctgaacatgctgacaagcgaggacacggccgtctattactgtgcaggaagcttggagtttagtggctggggagttatgcgctacggtataaactactggggccagggtatcctggtcaccgtctcg

SEQ ID NO: 43 - DNA, 375 bp, Equus caballus
caggtgcaactgcaggagtcaggacctggcctggtgaagccctcgcagaccctctccctcacctgcactgtctctggattatctttgagcagtaatgctgtaggctgggtccgccaggctccaggaaaagggctggaatatgttgatagtataggcaacagtgaaagtgcaaactttaacccagccctgaagtcccgagccagcatcaccgaggacacctcaaagagccgagtttatctgacgctgaacagcctgacaagcgaggacacggccgtctattactgtgcagcccaatatgattactttgctggtgcttatggcctcatcccttatgctataaagtactggggccagggtatcctggtcaccgtctcg

SEQ ID NO: 44 - DNA, 342 bp, Equus caballus
caggtgcaactgaaggagtcgggacctggcctggtgaagccctcgcagaccctgtccctcacctgcactgtctctggattccctttgagcagttacggtgtaggctgggtccgccaggctccaggaaaagggctggaatcggttggtgaaatagctagtagtggaagtgcaaactacaacccagccctgaagtcccgagccagcatcaccaaggacacctcaaagagccaagtttatctgacgctgaacagcctgacaagcgaggacacggccgtctattattgtacaggatggggactgagactgtactactggggccagggtatcctggtcaccgtctcg

SEQ ID NO: 45 - DNA, 369 bp, Equus caballus
caggtgcagctgcaggagtcaggacctggcctggtgaagccctcgcagaccctctccctcacctgtactgtctctggattatcactgagcagtaatgtgttaggctgggtccgccaggctcccggaaaagggctggaatggattggtggaatatatggaagtgcaagtccaaactataatctaaccctgaaggcccgaggcagcatcaccaaggacacctcaaagagccaagtgtatctgacgctaactgggatgacagaggaggacacggccgtctattactgtgcaggaggggctccctataattatgccggtggtaacattggaagaatgaagtattggggccagggtatcctggtcaccgtctcg

SEQ ID NO: 46 - DNA, 366 bp, Equus caballus
caggtgcagctgaaggagtcaggacctggcctggtgaagccctcgcagaccctctccctcacctgcactgtctctggattatctttgagcagttatggtgtgggctgggtccgccaggctccaggaaaaggtctggaatttgttggcggtatacttagtagtggaagggcaaactacaacccagccctgaagtcccgagccagcatcaccagggatacaacaaagaaccaagtttatctgacgctgaacagcctgacaggcgaggacacgtccgtctattactgtgcgagatcatttgctagtggtggttcttactacgactatgcgataaacttctggggccagggtatcctggtcaccgtctcg

SEQ ID NO: 47 - DNA, 336 bp, Equus caballus
caggtgcagctgaaggagtcaggacctggcctggtgaagccctcgcagaccctctccctcacctgcgctgtctctggattacctttgagagatgctgccgtaggctgggtccgccaggctccaggaaagggtctggaatatattggttctatgtataatgaagaagactacaatccagacctgaagtcccgagccagcgtcaccaaggacacctcaaagagccgagtcactctgacgctgaacagtctgacaagtgaggacacggccgtctattactgtgtaggggacggtggctctggatactactggggccagggtatcctggtcaccgtctcg

SEQ ID NO: 48 - DNA, 372 bp, Equus caballus
caggtgcaactgcaggagtcgggcccaggacaggtgaagccctcacagaccctctccctcacctgcactgtcactggaggatccatcacaaacaagtattctagctggacctggttacgccagcctccagggaagggcctggaatttatcggatacatatattatgatggtagacgttactacaatccttccttcaagagccgcacctccatctccagagacacctccaggaacgagttctccctgcagctgagctccgtgaccgatgaggacgcggccgtatatttttgtgcaggggattatggttatggcggtgtttggtactcagatggtgaaaactactggggccagggtatcctggtcaccgtctcg

SEQ ID NO: 49 - protein, 120 aa, Equus caballus
  misc_feature: clone 1-2
QVQLKESGPGLVKPSQTLSLTCTVSGLSVSSNGVAWVRQAPGKGLEFVGVIHTDGGVDYNPALKSRGSITRDISKSQLYLTLNTLTGEDTAVYYCARHASTGAYLYPFDYWGQGILVTVS

SEQ ID NO: 50 - protein, 114 aa, Equus caballus
  misc_feature: clone 1-2
QVQLKESGPGLVKPSQTLSLTCTVSGFSLNTYAVGWVRQAPGKGLEFVGSIYSIGSATYNLDLKSRVSITKDTSKSQVYLTVNSLTSEDTAVYYCGRRVNEIDYWGQGILVTVS

SEQ ID NO: 51 - protein, 123 aa, Equus caballus
  misc_feature: clone 1-4
QVQLKESGPGLVKPSQTLSLTCTVSGSSSEGYGVGWVRQAPGRGLEFVGGITNSGSARFNPGLASRASILKNTEKSQVYLTLTDLTGEDTAVYYCAKDSESGFLYWGHYGVEYWGQGILVTVS

SEQ ID NO: 52 - protein, 125 aa, Equus caballus
  misc_feature: clone 1-5
QVQLQESGPGLVKPSQTLSLTCTVSGLSLSSNTVGWVRQAPGKGLEYVGAIYGSASAAYNPALKSRASITKDTSKSQVYLTLNSLTSEDTAVYYCAGGGGGWIGYDYLGYYDINYWGQGILVTVS

SEQ ID NO: 53 - protein, 117 aa, Equus caballus
  misc_feature: clone 1-7
QVQLKESGPGLVKPSQTLSLTCTVSGLSDNSNAVGWVRQAPGKGLEFVADLTDSDSNPALKSRVRITKEPSKSQVRLIMNSLTEEDTAVYYCIHGYYNSFMVGAIKYWGQGILVTVS

SEQ ID NO: 54 - protein, 126 aa, Equus caballus
  misc_feature: clone 1-10
QVQLKESGPGLVKSSQTLSLTCTVSGASLNDIAVGWVRQAPGKGLEYVGCVYDGTGENYNPALKSRASITRDTSKSQVYLALNSLTSEDTAVYYCTGGKGDYGRYWNSYAEDGITNWGQGILVTVS

SEQ ID NO: 55 - protein, 126 aa, Equus caballus
  misc_feature: clone 1-11
QVQLKESGPGLVKPSQTLSLTCTVSGFSLITDSVGWVRQAPGKGLEFVGGLSSFGSANYNPGLNSRASITKDTSKGQVVLTLNSLTSDDTAVYYCVSFSGQGEAFAFAYLYYGITYWGQGILVTVS

SEQ ID NO: 56 - protein, 115 aa, Equus caballus
  misc_feature: clone 1-13
QVQLQESGPGLVMPSQTLSLTCTVSGLSLVNRNAVRWVRQAPGKGLEYVGSIYGVEERNYNPVLKSRVDITKDTSKSQVYLTLNSVTSGDTAVYYCARNEYGIVEWGQGILVTVS

SEQ ID NO: 57 - protein, 121 aa, Equus caballus
  misc_feature: clone 1-14
QVQLKESGPGLVKPSQTLSLTCTVSGLSLSSNTVGWVRQAPGKGLEYVGIIYGSASTLYNPALKSRASITKESSKSQVYLTLNSLTSEDTAVYYCAGGFSGFDWFDRGINYWGQGILVTVS

SEQ ID NO: 58 - protein, 123 aa, Equus caballus
  misc_feature: clone 2-1
QVQLKESGPGLVKPSQTLSLTCSVSGLNLNEDIVGWVRQAPGKGPEYVGSIWGDRSPKYNPDVKSRASISKDTSKRQVYLQLNSLSDEDTAVYYCAGGLTILGVMKDETFVDHWGPGILVTVS

SEQ ID NO: 59 - protein, 121 aa, Equus caballus
  misc_feature: clone 2-3
QVQLKESGPGLVKPSQTLSLTCTISGLSLSSYGVGWVRQAPGKGLEFVGGIRSSGSANYNPALKSRASITKDTSQSHVYLTLNSLTSEDTAVYYCAGGTEQRDYIDVGVKFWGQGILVTVS

SEQ ID NO: 60 - protein, 113 aa, Equus caballus
  misc_feature: clone 2-4
QVQLKESGPGLVKPSQTLSLTCTVSGLSLSSVDVGWVRQAPGKGLEYVSWIGRSTSYKPALKSRASITKDTSKSQAYLTLNSLTSEDTAVYYCVGGYADGIDYWGQGILVTVS

SEQ ID NO: 61 - protein, 121 aa, Equus caballus
QVQLKESGPGLVKISQTLSLTCTVSGLSLSSNDVGWVRQAPGKGLEYVARIWGGANEHYNPALKSRASITKDTSKSQVYLTLNSLTSEDTAVYYCGGTPGFYNSAYETFAYWGQGILVTVS

SEQ ID NO: 62 - protein, 119 aa, Equus caballus
  misc_feature: clone 2-6
QVQLKESGPGLVKPSQTLSLTCTVSGLLLNSNCVGWVRQAPGKRLEYVGSIYGTLTNYNSALRSRARITSDYSKSQVLLTLNSLTSEDTAVYYCAALDYGVTISRDINDWGQGILVTVS

SEQ ID NO: 63 - protein, 114 aa, Equus caballus
  misc_feature: clone 2-7
QVQLKESGPGLVKPSQTLSLTCTVSGLSLTGSQIAWVRQAPGKGLEYISGSSMYNPALKFRASITKDTSKNQVTLTLNKLTGEDTAVYYCVATAFWGGYGGIQYWGQGILVTVS

SEQ ID NO: 64 - protein, 122 aa, Equus caballus
  misc_feature: clone 2-8
QVQLKESGPGLVKPSQTLSLTCTVSGLSLTDYGVGWVRQAPGKGLEFVARIDSDGSKNFNPALKSRANIIKDTSKSQVYLTLNSLTSEDTAVYYCAGYGYSGRYSTPGNLYWWGQGILVTVS

SEQ ID NO: 65 - protein, 118 aa, Equus caballus
  misc_feature: clone 2-9
QVQLKESGPGLVKPSQTLSLVCTVSGFSLTDRGVGWVRQAPGKGLEFVSYILTSGAQDGNPALRSRVSITRDTSLSQVYLTMNSVTGEDTAVYYCGRHGPNLHGTFDYWGQGILVTVS

SEQ ID NO: 66 - protein, 123 aa, Equus caballus
  misc_feature: clone 2-11
QVQLKESGPDLMKPSQTLSLTCTVSGFSLSSYGVGWVRQAPGKGLEFVGGLPGSGSADYSPALRSRASITKDTSKSQVYVTLNSLTSEDTAVYYCARFYNWNSGVVSYTGIDYWGQGILVTVS

SEQ ID NO: 67 - protein, 124 aa, Equus caballus
  misc_feature: clone 2-14
QVQLKESGPGLVKPSQTLSLTCTVSGLSLSSYGVDWVRQAPGKGLEWVGGITSSGGSGYNPALKSRASITKDTSKSQVYLTLNSLTSEDTAVYYCAGEEEGYVYGFTRYYGNYYWGQGILVTVS

SEQ ID NO: 68 - protein, 121 aa, Equus caballus
  misc_feature: clone 2-15
QVQLQESGPGLVKPSQTLSLTCSVSGLSLSSVFVYWVRQAPGKGLEYVGFIGNSGSTIGNSGKTNYNYNPVLKSRASISKDTSKSQVLLTLNSLTSEDTAVYYCAGDNIKYWGQGILVTVS

SEQ ID NO: 69 - protein, 123 aa, Equus caballus
  misc_feature: clone 3-1
QVQLKESGPGLVKPSQTLSLICTVSGFSLSSDSVGWVRQAPGKGLEFVGVVHSSGRARNPALKSRASITKDTSESQVYLTLNSLTSEDTAVYYCAGGRSGYSYYAGMVDGINYWGQGILVTVS

SEQ ID NO: 70 - protein, 122 aa, Equus caballus
  misc_feature: clone 3-2
QVQLKESGPDLVKPSETLSLVCSVSGQSLSSYDVGWVRQAPGWGLEFVGVTAHYGGIDYNPALKSRASITKDTSKNQLTLILNSLTSEDTAVYYCTGEAQTNCDFGVSCLGYWGQGILVTVS

SEQ ID NO: 71 - protein, 119 aa, Equus cabalus
  misc_feature: clone 3-3
QVQLKESGPGLVKPSQTLSLTCTVSGLSLSSYGAGWVRQSPGKGLEYVGGVGKSGSSNYNSALKPRASITKDSSKSQISLTLRSLTGEDTAVYYCAIYDSYLRGWSVVYWGQGILVTVS

SEQ ID NO: 72 - protein, 121 aa, Equus caballus
  misc_feature: clone 3-4
QVQLKESGPGLVKPSQTLSLTCTVSGLSLRGNVVGWVRQAPGKGLEHVGENVSSGGAFYSPALKSRASITRDTSKSQIYLTLNSLTREDTAVYYCAAWKVSSRSYLDGINYWGQGILVTVS

SEQ ID NO: 73 - protein, 112 aa, Equus caballus
  misc_feature: clone 3-5
QVQLKESGPGLVKPSQTLSLTCAVSGFSLSSDGINWVRQAPGKGLEFVGSIYTSASTIYNPALKSRASITKDTSKSQVYLTLNSLTSEDTAVYYCSGGSEEYWGQGILVTVS

SEQ ID NO: 74 - protein, 124 aa, Equus caballus
  misc_feature: clone 3-6
QVQLQESGPGLVRPAETLSLTCTVSGLDLSSGTIIWVRQAPGKGLERVGEIVGEGSGFYNPALKSRAMITKDTSKNEIYLTLKSLTSEDTAVYYCAGAWGGNYYENFFINGVENWGQGILVTVS

SEQ ID NO: 75 - protein, 124 aa, Equus caballus
  misc_feature: clone 3-7
QVQLKESGPGLVKPSQTLSLTCTVSGLSLSSSCVQWVRQVPGKGLEYVGRIVSSGGGLTYNPALKSRASITRDTSKSQVYLTLNSLTDEDTAVYYCTGALNTHYSSYAGYGIDYWGQGILVTVS

SEQ ID NO: 76 - protein, 124 aa, Equus caballus
  misc_feature: clone 3-8
QVQLKESGPGLVKPAQTLTLTCTVSGLHLNSDAVVGWVRQAPGKGLEFVGGLSNTGRANYNPALKSRAIITKDTSKSQVYLTLNSLTSEDTADYFCAGGRMFDYVYGGYYEIQYWGQGILVTVS

SEQ ID NO: 77 - protein, 129 aa, Equus caballus
  misc_feature: clone 3-9
QVQLKESGPGLVKPSQALSLTCTISGFSLTSHGVGWVRQAPGKGLEFVGSIWTTGQTINNPTLKSRVSITRDTGLNQVSLTLNELTSEDTAVYYCAGGAISDYDFFGFRGMFSIYDVQYWGQGILVTVS

SEQ ID NO: 78 - protein, 122 aa, Equus caballus
  misc_feature: clone 3-12
QVQLKESGPGLVKPSQTLSLVCTVSGFSLNSWGVGWVRQAPGKGLEEVGGSQIGGNANYNPALESRASITKDASKSQVYLTLNSLTEEDTAVYYCTGGYNWNLGTNRDRITYWGQGILVTVS

SEQ ID NO: 79 - protein, 127 aa, Equus caballus
  misc_feature: clone 3-13
QVQLKESGPGLVKPGQTLSLSCTVSGLSLSTNTVGWVRQAPGKGWEYVAALYADADGDYNPVLQSRASITKDTSKNQVFLTLDTLTSEDTAVYYCTGGVFSVPVGTGYTYYESGILYWGQGILVTVS

SEQ ID NO: 80 - protein, 121 aa, Equus caballus
  misc_feature: clone 3-15
QVQLKESGPGLVNPSQTLSLTCFVSGFSLTSWHVGWVRQAPGKGLEFVGGIPVIGEAYYNPVLKSRISITKDTSKSQVYLTLNSLTDEDTAVYACARLRNWYGDYYSDMDYWGQGILVTVS

SEQ ID NO: 81 - protein, 114 aa, Equus caballus
  misc_feature: clone 4-1
QVQLQESGPGLVKPSQTLSLTCTVSGLSLNSYDVNWVRQAPGKGLEVVGSISDSGIAVYNPALKSRASITKDTSNSQVYLTLNSLTGEDTAVYYCARGNFAFDYWGQGILVTVS

SEQ ID NO: 82 - protein, 112 aa, Equus caballus
  misc_feature: clone 4-2
QVQLKESGPGLVKPSQTLSLTCAVSGFSLRDAAMGWVRQAPGRGLEYIGSMYIREDYNPALKSRASVTKDTKESRSYLTLNALTSEDTAVYWCVGDVGTGYYWGQGILVTVS

SEQ ID NO: 83 - protein, 119 aa, Equus caballus
QVQLKESGPGLVKPSQTLSLTCTVSGFSLSTTGVGWVRQAPGKGLEFVGGVPSSGSANYNPALKSRCSITKDESKSQVYLTLNSLTSEDTAVYICAGGFYNTLDKGINYWGQGILVTVS

SEQ ID NO: 84 - protein, 119 aa, Equus caballus
  misc_feature: clone 4-4
QVQLKESGPGLVKPSQTLSLTCTISGFSLTSASVDWVRQAPGKGLEFVGGIATSGRANYNPVLKSRATITRDTSKSQVYLTLNSLTGEDTAVYYCAESYYDGVGGNYYFWGQGILVTVS

SEQ ID NO: 85 - protein, 126 aa, Equus caballus
  misc_feature: clone 4-5
QVQLKESGPGLVKPSQTLSLTCTVSGMSLSTNTVGWVRQAPGKGLEYVGLIYGMKSAEYNPALKSRASITKDTSNSQVLLTLNSLTSEDTAVYYCAGGEAWGPMYSSNEEKNGVEYWGQGILVTVS

SEQ ID NO: 86 - protein, 117 aa, Equus caballus
  misc_feature: clone 4-6
QVQLQESGPGLVKPSQTLSLTCTVSGISLTDYNVDWVRQAPGKGLEFVGGLWTNGQSNYNPALKSRARITKDTSKSQVYLTLNSLTSEDTAVYYCEGYGNSWQPPHYWGQGILVTVS

SEQ ID NO: 87 - protein, 116 aa, Equus caballus
  misc_feature: clone 4-7
QVQLKESGPGLVKPSQTLSLTCTVSGNDLRSFGVAWVRQAPGKGLEFVGGVARFGSPYYNPALKSRAIITKDTSKKESVLTLNSVTGEDTAVYWCAGGYGDESWGPWGQGILVTVS

SEQ ID NO: 88 - protein, 123 aa, Equus caballus
  misc_feature: clone 4-8
QVQLKESGPGLVKPSQTLSLTCTVSALSLSSAGVGWVRQAPGKGLEFVAGIVGDGGTYANPALRSRASITKDTSKSQVYLTLNMLTSEDTAVYYCAGSLEFSGWGVMRYGINYWGQGILVTVS

SEQ ID NO: 89 - protein, 125 aa, Equus caballus
  misc_feature: clone 4-9
QVQLQESGPGLVKPSQTLSLTCTVSGLSLSSNAVGWVRQAPGKGLEYVDSIGNSESANFNPALKSRASITEDTSKSRVYLTLNSLTSEDTAVYYCAAQYDYFAGAYGLIPYAIKYWGQGILVTVS

SEQ ID NO: 90 - protein, 114 aa, Equus caballus
  misc_feature: clone 4-10
QVQLKESGPGLVKPSQTLSLTCTVSGFPLSSYGVGWVRQAPGKGLESVGEIASSGSANYNPALKSRASITKDTSKSQVYLTLNSLTSEDTAVYYCTGWGLRLYYWGQGILVTVS

SEQ ID NO: 91 - protein, 123 aa, Equus caballus
  misc_feature: clone 4-11
QVQLQESGPGLVKPSQTLSLTCTVSGLSLSSNVLGWVRQAPGKGLEWIGGIYGSASPNYNLTLKARGSITKDTSKSQVYLTLTGMTEEDTAVYYCAGGAPYNYAGGNIGRMKYWGQGILVTVS

SEQ ID NO: 92 - protein, 122 aa, Equus caballus
  misc_feature: clone 4-12
QVQLKESGPGLVKPSQTLSLTCTVSGLSLSSYGVGWVRQAPGKGLEFVGGILSSGRANYNPALKSRASITRDTTKNQVYLTLNSLTGEDTSVYYCARSFASGGSYYDYAINFWGQGILVTVS

SEQ ID NO: 93 - protein, 112 aa, Equus caballus
  misc_feature: clone 4-14
QVQLKESGPGLVKPSQTLSLTCAVSGLPLRDAAVGWVRQAPGKGLEYIGSMYNEEDYNPDLKSRASVTKDTSKSRVTLTLNSLTSEDTAVYYCVGDGGSGYYWGQGILVTVS

SEQ ID NO: 94 - protein, 124 aa, Equus caballus
  misc_feature: clone 4-15
QVQLQESGPGQVKPSQTLSLTCTVTGGSITNKYSSWTWLRQPPGKGLEFIGYIYYDGRRYYNPSFKSRTSISRDTSRNEFSLQLSSVTDEDAAVYFCAGDYGYGGVWYSDGENYWGQGILVTVS

SEQ ID NO: 95 - DNA, 19 bp, Artificial sequence
  Synthetic primer
gtccaccttggtgctgctg

SEQ ID NO: 96 - DNA, 22 bp, Artificial sequence
  Synthetic primer
caggtgcarctgmaggagtcrg

SEQ ID NO: 97 - DNA, 36 bp, Artificial Sequence
  Synthetic primer
gcctccaccactcgagacggtgaccaggataccctg

SEQ ID NO: 98 - DNA, 49 bp, Artificial sequence
  Synthetic primer
ttactcgcggcccagccggccatggcccaggtgcarctgmaggagtcrg

(19) United States
(12) Patent Application Publication
(10) Pub. No.: US 2009/0155850 A1
(43) Pub. Date: Jun. 18, 2009

(54) Horse:Human Chimeric Antibodies

(75) Inventors: Juan C. Almagro, Haverford, PA (US); Alejandro Alagon-Cano, Cuernavaca (MX); Jorge P. Solis, Tlalpan (MX); Sylvia L. Smith, Miami, FL (US); Alvaro Velandia, Miami, FL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: The Florida International University Board of Trustees, Miami, FL (US)

(21) Appl. No.: 12/090,166

(22) PCT Filed: Oct. 27, 2006
(86) PCT No.: PCT/US06/42236

§ 371 Date: Nov. 24, 2008

Related U.S. Application Data
(60) Provisional application No. 60/731,185, filed on Oct. 28, 2005.

(51) Int. Cl. C12P 21/00; C07K 16/18; C12N 9/96

(52) U.S. Cl. 435/69.6; 530/387.3; 530/391.3; 530/391.7; 435/188

(57) ABSTRACT

The present invention provides a plurality of chimeric single chain variable region (scFv) antibodies. The chimeric scFv antibodies individually comprise variable regions from both horse and non-horse antibodies. Methods of making and using the plurality are also provided.

### CROSS-REFERENCE TO RELATED APPLICATIONS

This application is based on U.S. application Ser. No. 60/731,185, filed Oct. 28, 2005, the disclosure of which is incorporated herein in its entirety.

### BACKGROUND

Antibodies are globular proteins present in the blood serum. These proteins, also known as immunoglobulins (Igs), play a crucial role in the adaptive immunity. They recognize non-self antigens and neutralize them and/or facilitate their elimination. These immune receptors thus evolved to recognize any other molecule with exquisite specificity and high affinity, which has proven to be of great potential in molecular biology, clinical diagnostic research, proteomics and therapeutic applications.

Of the known types of Igs, namely IgG, IgM, IgD, IgA and IgE, IgG is the most abundant in the blood circulation. IgGs are the product of an immune response maturation and therefore are highly specific and in general high affinity antibodies. As a result, the vast majority of antibodies that are commercially produced belong to the IgG type.

All the IgGs have the same general structure. They are composed of two identical polypeptide heavy (H) chains and two identical polypeptide light (L) chains. Each H chain has one variable (V_{H}) domain and three constant domains, CH1, CH2, and CH3, counted from the V_{H} domain at the amino terminal end. The L chain has one variable domain (V_{L}) at the amino terminal end and only one constant domain, C_{L}.

The V_{H} and CH1 domains of one H chain associate with the V_{L} and C_{L} domains of one L chain to form an antigen-binding fragment (Fab). The CH2 and CH3 domains from one H chain associate with the CH2 and CH3 domains from the other H chain to form the crystallizing fragment (Fc). This fragment connects two Fabs via the hinge segments between CH1 and CH2, giving to the IgG molecule its typical “Y” shape.

Each V domain is composed of four conserved framework regions (FW-1 to FW-4) that alternate with three loops that vary in length and amino acid composition. These hypervariable loops, or complementary determining regions (CDRs), denoted CDR-H1, CDR-H2 and CDR-H3 for V_{H}, and CDR-L1, CDR-L2 and CDR-L3 for V_{L}, are brought together by non-covalent association of the V_{H} and V_{L} domains in a Fv fragment, and form the antigen-binding site at the terminus of the Fab fragment.

When an IgG is digested enzymatically, different fragments are obtained depending on the enzyme used. That is, if papain is used, three fragments are obtained: one Fc fragment and two Fabs. If pepsin is used, two fragments are obtained: one Fc fragment and one F(ab′)₂ fragment. The foregoing is due to the fact that papain cuts the H chains in the hinge region before a disulfide bridge, while pepsin cuts them in the hinge region after the disulfide bridge. Therefore, papain digestion releases two Fab fragments, while pepsin leaves the two Fab fragments bound via the disulfide bridge. Fab and F(ab′)₂ fragments conserve their capacity to specifically bind to the antigen against which they were produced, as they contain the Fv fragment.

When one species is administered whole antibodies elicited in another species, the former generates an immune response against antigenic determinants of the latter. This result may give rise to varied adverse secondary responses that can even include anaphylactic shock. These problems are significantly reduced when the antibodies are previously digested with papain or pepsin and only the resulting purified Fab or F(ab′)₂ fragments are administered. The use of F(ab′)₂ fragments has a particular advantage over the use of Fab fragments in that they are retained far longer in the organism. Moreover, because F(ab′)₂ fragments have two Fabs, they are able to form a network that precipitates the antigen in physiological conditions.

Because F(ab′)₂ fragments conserve the main characteristics of intact antibodies, the applications of the antibodies extend to F(ab′)₂ fragments, with the additional advantage, that because they lack the Fc fragment, recognition as foreign by a patient to whom they are administered is less likely. This result provides greater tolerance to application of F(ab′)₂ fragments and reduces the possibility of secondary reactions.

In some applications, the use of Fv fragments has advantages over Fab and F(ab′)₂ fragments. In the particular case of acute envenomation or intoxication, faster clearance times are desirable. The Fv fragment is half the molecular weight of the Fab fragment, and is eliminated from the organism—together with the toxin or drug—faster. Fv fragments are easily produced and purified by recombinant technologies as scFv (single chain Fv fragments).

Antibody genes, or fragments thereof, once isolated can be modified through molecular biology techniques. This possibility offers additional advantages, such as the modification of chemico-physical properties of antibodies to obtain more stable therapeutics, or grafting the antigen-binding site of a non-human antibody into a human framework to produce less immunogenic molecules, or maturating in vitro the affinity of the antibody for the antigenic determinant to reach affinities that cannot be obtained in vivo.

In regions where, due to climatic conditions, venomous animals abound, antibodies have been given a special use to combat venom toxicity. In general, a large number of doses are administered when treating patients with scorpion, spider and snake stings or bites. For example, the therapy of choice in cases of scorpion envenomation in humans is the intravenous administration of highly purified F(ab′)₂ fragments obtained by pepsin digestion of the IgG fraction produced by horses (Equus caballus) after immunization with extract of the venom glands from four scorpion species of the genus Centruroides. Although the resulting product is highly effective, the fact that the therapeutic is still composed of V and C domains heterologous to humans, it can elicit human anti-horse immune responses. Furthermore, preparation and quality control of this product requires large number of animals, including horses, mice and scorpions, which is costly and ethically questionable.

Thus, there continues to exist a need in the art for new therapeutics useful in treatment regimens which reduce the likelihood of evoking an immune response in the recipient of the treatment. New therapeutics which can be produced by recombinant technology would also be of increased economic value and would circumvent the need for using live animals as production sources.

### SUMMARY OF THE INVENTION

The present invention provides a plurality of chimeric scFv antibodies comprising at least two or more chimeric scFv antibodies that are immunospecific for different/distinct epitopes, said chimeric scFv antibodies individually comprising a first V domain derived from a horse and a second V domain derived from a species which is not a horse (“non-horse”). In one embodiment, the second V domain is derived from a human. In one aspect, the plurality of chimeric scFv antibodies is biased toward immunospecific recognition of toxin epitopes. In another aspect, the toxin is a neurotoxin.

In one embodiment, a plurality of chimeric scFv antibodies is provided wherein each of the horse V domains is a V_{H} fragment and each of the non-horse V domains is a V_{L} fragment. In one aspect, each non-horse V domain in the plurality is identical. In another aspect, the non-horse V domains are a human V domain, and in yet another aspect, each of the human V domains in the plurality is V_{L} fragment A27/Jk1 (SEQ ID NO: 2). In another embodiment, each of the horse V domains is a V_{L} fragment and each of the non-horse V domains is a V_{H} fragment. In one aspect, each non-horse V domain in the plurality is identical as described above.

“V_{H} fragment” as used herein refers to the heavy chain variable region of an antibody comprising at least one CDR of an antibody heavy chain variable domain. The V_{H} chain may contain one, two, or three CDRs of an antibody V_{H} chain, designated as H1, H2 and H3 fragments.

“V_{L} fragment” as used herein refers to the light chain variable region of an antibody comprising at least one CDR of an antibody light chain variable domain. The V_{L} chain may contain one, two, or three CDRs of the antibody light chain, which may be either a kappa or lambda light chain depending on the antibody. The CDRs in the light chain variable region are designated as L1, L2 and L3 fragments.

The invention further provides a plurality of chimeric scFv antibodies wherein the either the V_{H} fragment or the V_{L} fragment is selected from a phage display library.

In one embodiment, the plurality of chimeric scFv antibodies of the invention includes a V_{H} fragment which comprises one or more fragments selected from the group consisting of an H1 fragment, an H2 fragment, and an H3 fragment. In another aspect, the plurality of chimeric scFv antibodies of the invention includes a V_{H} fragment which comprises an H1 fragment and an H2 fragment, the V_{H} fragment comprises an H1 fragment and an H3 fragment, the V_{H} fragment comprises an H2 fragment and an H3 fragment or the V_{H} fragment comprises an H1 fragment, an H2 fragment and an H3 fragment.

In another embodiment, the plurality of chimeric scFv antibodies of the invention include a V_{L} fragment which comprises one or more fragments selected from the group consisting of an L1 fragment, an L2 fragment and an L3 fragment. In various aspects, the plurality of chimeric scFv antibodies of the invention includes a V_{L} fragment which is an L1 fragment, an L2 fragment, an L3 fragment, an L1 fragment and an L2 fragment, an L2 fragment and an L3 fragment, or an L1 fragment, an L2 fragment and an L3 fragment.

The invention further provides a plurality of chimeric scFv antibodies which comprises one or members of the plurality having one or more natural or non-natural modifications which do not eradicate the affinity of said chimeric scFv antibodies to an epitope. In one aspect, the one or more natural or non-natural modifications is selected from the group consisting of deletion, insertion, substitution and covalent modification to include a protein or non-protein moiety.

The invention further provides a plurality of chimeric scFv antibodies wherein one or more of the chimeric scFv antibodies are conjugated to a second polypeptide. In one aspect, the second polypeptide is a fragment of a second antibody. In another aspect, the invention provides a plurality of chimeric scFv antibodies wherein the chimeric scFv antibodies are conjugated to a water soluble polymer, and in one aspect, the water soluble polymer is polyethylene glycol.

In another aspect, the plurality of chimeric scFv antibodies are labeled, and in various embodiments, the label is selected from the group consisting of enzymes, radioisotopes and fluorescent compounds.

The invention also provides methods of mutagenesis of a plurality of chimeric scFv antibodies of the invention comprising: a) mutagenizing genes encoding the individual chimeric scFv antibodies; and b) expressing the genes to produce mutagenized chimeric scFv antibodies. In another aspect, methods of the invention further comprise the step of screening the mutagenized chimeric scFv antibodies to select for a desired structure or function. In one embodiment, mutagenizing in a method of the invention is accomplished by site-directed mutagenesis.

### BRIEF DESCRIPTION OF THE FIGURES

FIG. 1: Schematic depiction of phagemid vector pHEN-A27.

### DETAILED DESCRIPTION OF THE INVENTION

The present invention provides a plurality of chimeric scFv antibodies and materials and methods for making and using the same. A plurality of scFv antibodies of this type is particularly useful for treatment of conditions which arise in many species but for which vaccination is not possible due, at least in part, to a lack of approved and/or effective vaccination. Certain conditions of this type afflict horses as a normal course of environmental contact and in these instances, many horses have developed specific immunity, in part in the form of specific antibody production, making these horses resistant to complications associated with the conditions that would otherwise manifest in species which have not been subject to the same environmental challenges. By producing a plurality of chimeric antibodies which comprise a horse variable region fragment and a variable region fragment from a non-horse species, one or more members of the plurality can be identified and utilized for passive immunization of the non-horse species in the treatment of a condition for which the horse variable region would be therapeutically beneficial. By having the non-horse variable region component of the scFv being derived from the species receiving the therapeutic treatment, the treatment regimen in less likely to evoke an anti-horse antibody response as would be a potential problem if both variable regions in the scFv were derived from a horse.

In one aspect, the plurality of chimeric scFv antibodies comprises at least two or more chimeric scFv antibodies that are immunospecific for different/distinct epitopes. Because the individual scFv antibodies in the plurality comprise a horse V domain and a non-horse V domain, it will be readily understood that the horse and non-horse V domains will in most cases, but not always, specifically bind to distinct epitopes. However, “immunospecificity for different/distinct epitopes” as used herein means that the individual horse V components in the scFv antibodies of the plurality specifically bind to distinct epitopes compared to each other and not compared to binding of non-horse V domain(s).

“Specifically binds” or “immunospecificity” as used herein means that a V domain (either horse or non-horse) of an scFv antibody in the plurality preferentially binds a single and specific epitope at least 10×, at least 100×, or at least 1000× greater than other epitopes when both epitopes are available in equal amounts. Thus, a V domain of an antibody in the plurality may cross-react with (or bind to) multiple epitopes, but to a degree and with an affinity that is insignificant compared to a single epitope against which the V domain was generated.

The phrase “biased toward immunospecific recognition” as used herein means that a higher number or a higher percentage of chimeric scFv antibodies in the plurality is immunospecific for a specific antigen or antigens compared to a randomly generated plurality of chimeric scFv antibodies. A higher number or a higher percentage of chimeric scFv antibodies in the plurality immunospecific for a specific antigen demonstrate, in various aspects, significantly greater immunospecific binding for a specific antigen(s) compared to a randomly generated plurality of chimeric scFv antibodies. The higher number or higher percentage, in various aspects, is about half (approximately 50%), a majority (>50%), essentially all (>85%) or all (100%) of the chimeric scFv antibodies in the plurality that are immunospecific for a specific antigen compared to a randomly generated plurality of chimeric scFv antibodies. In one aspect, the antigen is a toxin.

In various aspects, different horse V domains in the plurality of scFv antibodies will specifically bind to at least two different/distinct epitopes, at least five different/distinct epitopes, at least 10 different/distinct epitopes, at least 10² different/distinct epitopes, at least 10³ different/distinct epitopes, at least 10⁴ different/distinct epitopes, at least 10⁵ different/distinct epitopes, at least 10⁶ different/distinct epitopes, at least 10⁷ different/distinct epitopes, or more. In one aspect, two or more horse V domains in the plurality may specifically bind to the same epitope and yet the individual scFv antibodies may still differ in primary amino acid sequence, i.e., structurally distinct horse V domains may compete for binding to a single epitope.

In one embodiment, the non-horse V domain in each chimeric antibody of the plurality is identical. In this aspect, differences between individual chimeric antibodies in the plurality arise only from differences between primary amino acid sequence and/or specific binding properties of the horse V domains in the antibodies. In an alternative aspect, individual antibodies in the plurality may differ from each other by having unique non-horse V domains. That is not to say that, at least according to this aspect of the invention, an individual antibody in the plurality has more than one non-horse V domain itself, but instead the single non-horse V domain in each antibody need not be identical to all other non-horse V domains in the plurality. Thus, in various aspects, a plurality may include individual chimeric scFv antibodies having at least two, at least five, at least 10, at least 10², at least 10³, at least 10⁴, at least 10⁵, at least 10⁶, at least 10⁷, or more different non-horse V domains in the individual antibodies in the plurality.

The phrase “do not eradicate the affinity” with respect to the plurality of chimeric scFv antibodies comprising one or more natural or non-natural modifications means that the binding affinity of the chimeric scFv antibodies is not eliminated.

The phrase “do not substantially alter the affinity” with respect to the plurality of chimeric scFv antibodies comprising one or more natural or non-natural modifications means that the binding affinity of the plurality of chimeric scFv antibodies is not significantly increased or significantly decreased compared to the unmodified (wild type) plurality of chimeric scFv antibodies.

The phrase “natural or non-natural modifications” with respect to the plurality of chimeric scFv antibodies means an alteration to the amino acid sequences of the unmodified (wildtype) plurality of chimeric scFv antibodies. In one aspect, the modification is a deletion, insertion, or substitution of one or more amino acids of the amino acid sequences of the plurality of chimeric scFv antibodies with one or more naturally-occurring or non-naturally occurring amino acids. Natural modifications include amino acid changes in a wild type sequence with one or more amino acids that exist in nature including alanine, arginine, asparagine, aspartic acid (or aspartate), cysteine, glutamine, glutamic acid (or glutamate), glycine, histidine, isoleucine, leucine, lysine, methionine, phenylalanine, proline, serine, tryptophan, threonine, tyrosine and valine. Non-natural modifications include amino acid changes in a wild type sequence with one or more amino acids that do not exist in nature including β-alanine (β-aminopropionic acid), norleucine, norvaline, ornithine, N-methylvaline, N-methylisoleucine, N-methylglycine (carnosine), allo-isoleucine, 4-hydroxyproline, isodemosine, 3-hydroxyproline, allo-hydroxylysine, hydroxylysine, N-ethylasparagine, N-ethylglycine, 2,3-Diaminopropionic acid, 2,2′-diaminopimelic acid, demosine, 2,4-diaminobutyric acid, 2-aminopimelic acid, 3-aminoisobutyric acid, 2-aminoisobutyric acid, 2-aminoheptanoic acid, 6-aminocaproic acid, 4-aminobutyric acid (piperidinic acid), 2-aminobutyric acid, 3-aminoadipic acid and 2-aminoadipic acid. In another aspect, the non-natural modification includes the linking of the plurality of chimeric scFv antibodies to peptides, chemical agents or other agents compared to unmodified (wildtype) plurality of chimeric scFv antibodies.

Exemplary non-horse V domains can be derived from a variety of animals including, but not limited to, humans; farm animals such as cows, sheep, pigs, llamas and goats; companion animals such as dogs and cats; exotic and/or zoo animals; laboratory animals including mice, rats, rabbits, guinea pigs and hamsters; and poultry such as chickens, turkey, ducks and geese.

In various aspects of the invention, individual scFv antibodies in the plurality can comprise an intact and complete horse V domain, including one or more of the CDRs found in the V domain. Accordingly, in an instance wherein the individual chimeric scFv antibodies of the plurality comprise a horse V_{H} fragment, the individual members of the plurality can comprise CDR-H1, CDR-H2, and CDR-H3. Likewise, when the individual chimeric scFv antibodies comprise a horse V_{L} fragment, individual species in the plurality can comprise CDR-L1, CDR-L2, and CDR-L3. The amino acid sequences which connect the individual CDRs, i.e., the framework sequences, can be the naturally-occurring horse V domain framework sequences, or can be derived from other sources (including synthetic preparation) as discussed herein, as long as the resulting scFv antibody retains the specific epitope binding of the parental V domain into which the modifications are introduced. It is understood, however, that an antibody modified in the V framework region can have increased or decreased binding affinity for the specific epitope it recognizes.

In an alternative aspect, individual chimeric scFv antibodies of the plurality can comprise two V domain CDRs. For example, when the chimeric scFv antibody includes a V_{H} fragment, individual species can comprise a combination of CDRs H1 and H2, CDRs H1 and H3, or CDRs H2 and H3. The same is true in embodiments wherein the individual chimeric scFvs comprise a V_{L} fragment, species thus comprising a combination of CDRs L1 and L2, CDRs L1 and L3, and CDRs L2 and L3. Once again, the chimeric scFv antibodies modified to include only these CDRs from the parental V domain will retain the specificity for the epitope which is recognized by the V domain from which the individual CDRs were obtained, however, the binding affinity for the epitope may be modified.

In still another aspect, individual chimeric scFv antibodies in the plurality can comprise a single CDR from a horse V domain. If the chimeric scFv antibody includes a horse V_{H} fragment, that portion of the chimeric scFv antibody may comprise a single CDR H1, CDR H2, or CDR H3. If the chimeric scFv antibody comprises a horse V_{L} fragment, that portion of the chimeric scFv antibody may comprise a single CDR-L1, CDR-L2 or CDR-L3. As discussed above, chimeric scFv antibodies of these types retain epitope binding specificity of the parental V domain from which the individual CDRs were obtain, although the affinity of binding for the epitope may be modified.

These modified aspects of the individual antibodies in the plurality are also applicable to the non-horse V domain of the individual scFv antibodies. For example, the non-horse V domain may comprise CDR-H1, CDR-H2, and CDR-H3; CDR-L1, CDR-L2, and CDR-L3; CDRs H1 and H2; CDRs H1 and H3; CDRs H2 and H3; CDRs L1 and L2; CDRs L1 and L3; CDRs L2 and L3; CDR-H1; CDR-H2; CDR-H3; CDR-L1; CDR-L2; or CDR-L3 as long as the antibody modified in any of these ways retains the epitope binding specificity of the non-horse V domain from which the CDRs were obtained. Again, it is understood that the resulting scFv antibody may bind to the specific epitope with modified binding affinity.

Further modifications to the individual scFv antibodies, or groups of antibodies thereof, are discussed in further detail herein.

I. Production of Chimeric scFv Antibodies

ScFv antibodies can be generated by a number of methodologies that are readily available in the art. For example, scFv antibodies can be generated from hybridomas that express a monoclonal antibody having the desired antigen binding specificity and affinity. Oligonucleotides encoding antibody heavy and light chain variable domains may be amplified from total hybridoma cell RNA, wherein a polynucleotide encoding one of the V domains is amplified from one cell type and a polynucleotide encoding the other V domain is derived from a second cell type. Oligonucleotides encoding the individual heavy and light chain V domains may then be amplified from the cDNA by utilizing primer pairs that hybridize 5′ and 3′ to each of the heavy and light chain variable region coding regions. See, for example, U.S. Pat. Nos. 4,683,195, 4,683,202, and 4,800,159, the disclosures of which are incorporated herein in their entireties. Primer sequences suitable for PCR amplification of scFv antibody heavy and light chains are disclosed in U.S. Pat. No. 6,248,516 and PCT Patent Publication No. WO 90/05144.

Oligonucleotides encoding the individual heavy and light chain V domains isolated in this way may be combined by utilizing conventional recombinant DNA methodology such that the polynucleotide comprising the V_{H} coding region is fused in-frame with the polynucleotide comprising the V_{L} coding region. Depending on the precise scFv to be expressed, it may be desirable to ligate the V_{H} coding region 5′ to the V_{L} coding region. Alternatively, the V_{H} coding region may be ligated 3′ to the V_{L} coding region. Regardless of the orientation, in-frame ligation of the V_{H} and V_{L} coding regions permits translation into a single scFv protein that retains the biological activity of the component V_{H} and V_{L} polypeptides. For general guidance on the design of scFv antibodies, see U.S. Pat. No. 4,946,778, the disclosure of which is incorporated herein by reference in its entirety.

Other methods of producing scFv antibodies are described in Whitlow et al., Methods: A Companion to Methods in Enzymology 2:97 (1991); Bird et al., Science 242:423 (1988); and Pack et al., Bio/Technology 11:1271 (1993), all of which are incorporated herein by reference in their entireties.

Each of the above-described methodologies can be modified by those of skill in the art to incorporate horse V_{H} fragments and non-horse V_{L} fragments in order to produce chimeric scFv antibodies.

Chimeric scFv antibodies can also be generated by first immunizing an animal with an antigen or mixture of antigens that has been prepared for injection, with or without adjuvants. The antigens used for immunizing an animal can be any substance which is capable of inducing a specific immune response and of reacting with the products of that response, that is, with specific antibodies or specifically sensitized T-lymphocytes, or both. Antigens may be soluble substances, such as toxins and foreign (“non-self”) proteins, or particulates, such as bacteria and tissue cells. In other aspects, immunization may be unintentional, arising from environmental factors. While not true immunization in the art-accepted sense, introduction into an animal of environmental antigens that evoke an immune response provide a result similar to an intentional immunization, i.e., antibodies may be produced. Environmental factors that induce such an antibody response include dietary factors, atmospheric particulates, animal scratches and bites, and the like.

Nucleic acids encoding a protein antigen can also be used to immunize an animal. It has now been shown in a number of systems that direct injection of a nucleic acid can effectively immunize against the encoded product (U.S. Pat. Nos. 5,589,466 and 5,593,972; Hedley et al., Nature Med. 4:365-368, 1998; Ho et al., Arch. Virol. 143:115-125, 1998; Cardoso et al., J. Virol. 72:2516-2518.1998; Bagarazzi et al., Curr. Top. Microbiol. Immunol. 226:107-143, 1998; Lozes et al., Vaccine 15:830-833, 1997; Shiver et al., Vaccine 15:884-887, 1997, the disclosures of which are incorporated herein by reference in their entireties).

In one aspect of the invention, bias in the plurality of chimeric scFv antibodies towards immunospecific recognition of epitopes of a particular type can be induced. For example, bias can be created by immunizing an animal with a specific antigen or a mixture of antigens to evoke an immune response that include a significant number of individual antibodies that specifically bind to one or more epitopes on that particular antigen or antigens. Other methods of generating bias of antibodies towards specific antigens/epitopes are well known in the art (see, for example, U.S. Patent Application Publication No. 20030092125, the disclosure of which is incorporated by reference herein in its entirety).

At various times after immunization, blood samples are obtained from the animal for measurement of serum antibodies. Serum antibody titer is determined with various techniques known in the art, such as enzyme-linked immunosorbent assay (ELISA) and flow cytometry.

It will be understood that the above-described immunization protocol with an antigen or mixture of antigens, is not limited to a single injection, but may encompass immunization schedules that include both a primary and subsequent booster immunizations, with and without adjuvants, as is well understood in the immunologic arts.

Once circulating antibody titer reaches a desired level, the V domains of the antibodies can be cloned from hematopoietic cells of the immunized animal, sequenced and cloned by recombinant techniques as described herein or otherwise known in the art. For example, a cDNA library may be constructed by reverse transcription of cellular mRNA and the library screened using probes specific for immunoglobulin polypeptide gene sequences. In another embodiment, polymerase chain reaction (PCR) is used to amplify polynucleotides encoding immunoglobulin or fragments thereof. The amplified sequences can be readily cloned into any suitable vector, e.g., expression vectors, minigene vectors, or phage display vectors. In one aspect, the vector also encodes a variable region fragment from an antibody of a different mammalian species. The plurality of chimeric scFv antibodies is obtained after expressing and isolating the encoded proteins in an appropriate host cell.

It will be understood by those of skill in the art that the methods described above can be used to clone an individual V domain or a plurality of V domains as contemplated by the present invention. One such modification is described in the examples attached hereto. Even if the method of preparation is designed to produce only a single specific V domain, the method can be repeated to produce one or more alternative V domains to complete the desired plurality.

II. Production of Chimeric scFv Antibody Variants

Once a chimeric scFv antibody or even a plurality of scFv antibodies has been prepared, its physical, chemical and/or biological (immunological) properties can optionally be modified by altering one or more amino acid residues in its amino acid sequence and screening for changes in one or more properties. Amino acid sequence variants include substitution, deletion or insertion variants. In certain instances, variants are prepared with the intent to modify those amino acid residues which are directly involved in epitope binding. In other aspects, modification of residues which are not directly involved in epitope binding or residues not involved in epitope binding in any way, is desirable, for purposes discussed herein.

In order to determine which amino acid residues are important for epitope recognition and binding, alanine scanning mutagenesis can be performed to produce substitution variants. See, for example, Cunningham et al., Science, 244:1081-1085 (1989), the disclosure of which is incorporated herein by reference in its entirety. In this method, individual amino acid residues are replaced one-at-a-time with an alanine residue and the resulting scFv antibody screened for its ability to bind its specific epitope relative to the unmodified antibody. Those modified antibodies with reduced binding capacity are sequenced to determine which residue was changed, indicating its significance in binding. Alternatively, or in addition, it may be beneficial to analyze a crystal structure of the antigen-antibody complex to identify contact points between the chimeric scFv antibody and the antigen. Such contact residues and neighboring residues are candidates for substitution according to the techniques elaborated herein. Once such variants are generated, the panel of variants is subjected to screening as described herein and parent chimeric scFv antibodies with superior properties in one or more relevant assays may be selected for further development. Residues thus identified provide targets for numerous types of variants contemplated by the invention.

Substitution variants are those in which at least one residue in the antibody molecule amino acid sequence is removed and a different residue is inserted in its place. Substitution mutagenesis within any of the CDR regions and/or framework regions is contemplated. Modifications in the biological properties of the parent chimeric scFv antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. In certain aspects of the invention, substitution variants are designed, i.e., one or more specific (as opposed to random) amino acid residues are substituted with a specific amino acid residue. Typical changes of these types include conservative substitutions and/or substitution of one residue for another based on similar properties of the native and substituting residues.

Conservative substitutions are shown in Table 1. The most conservative substitution is found under the heading of “preferred substitutions.” If such substitutions result in no change in biological activity, then more substantial changes may be introduced and the products screened.

TABLE 1

| | | Preferred Residue |
|---|---|---|
| Original | Exemplary | Substitutions |
| Ala (A) | val; leu; ile | val |
| Arg (R) | lys; gln; asn | lys |
| Asn (N) | gln; his; asp, lys; gln | arg |
| Asp (D) | glu; asn | glu |
| Cys (C) | ser; ala | ser |
| Gln (Q) | asn; glu | asn |
| Glu (E) | asp; gln | asp |
| Gly (G) | ala | |
| His (H) | asn; gln; lys; arg | |
| Ile (I) | leu; val; met; ala; | leu |
| | phe; | norleucine |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | |
| Pro (P) | ala | |
| Ser (S) | thr | |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Amino acid residues which share common side-chain properties are often grouped as follows.

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

As an alternative to specifically designed substitution variants, other substitution variants can be prepared by affinity maturation wherein random amino acid changes are introduced into the parental antibody sequence. See, for example, Ouwehand et al., Vox Sang 74 (Suppl 2):223-232, 1998; Rader et al., Proc. Natl. Acad. Sci. USA 95:8910-8915, 1998; Dall'Acqua et al., Curr. Opin. Struct. Biol. 8:443-450, 1998, the disclosures of which are incorporated herein by reference in their entireties. Affinity maturation involves preparing and screening the chimeric scFv antibodies, or variants thereof and selecting from the resulting variants those that have modified biological properties, such as binding affinity relative to the parent chimeric scFv antibody. A convenient way for generating substitutional variants is affinity maturation using phage display. Briefly, several hypervariable region sites are mutated to generate all possible amino substitutions at each site. The variants thus generated are expressed in a monovalent fashion on the surface of filamentous phage particles as fusions to the gene III product of M13 packaged within each particle. The phage-displayed variants are then screened for their biological activity (e.g., binding affinity).

Techniques utilizing gene shuffling and directed evolution may also be used to prepare and screen chimeric scFv antibodies, or variants thereof, for desired activity. For example, Jermutus et al., Proc Natl Acad Sci USA., 98(1):75-80 (2001) showed that tailored in vitro selection strategies based on ribosome display were combined with in vitro diversification by DNA shuffling to evolve either the off-rate or thermodynamic stability of scFvs; Fermer et al., Tumour Biol. 2004 January-April; 25(1-2):7-13 reported that use of phage display in combination with DNA shuffling raised affinity by almost three orders of magnitude. Dougherty et al., Proc Natl Acad Sci USA. 2000 Feb. 29; 97(5):2029-2034 reported that (i) functional clones occur at an unexpectedly high frequency in hypermutated libraries, (ii) gain-of-function mutants are well represented in such libraries, and (iii) the majority of the scFv mutations leading to higher affinity correspond to residues distant from the binding site.

Deletion variants are polypeptides wherein at least one amino acid residue of a chimeric scFv antibody amino acid sequence is removed. Deletions can be effected at one or both termini of the protein, or with removal of one or more residues within (i.e., internal to) a chimeric scFv antibody amino acid sequence. Methods for preparation of deletion variants are routine in the art. See, e.g., Sambrook et al. (1989) Molecular Cloning: A Laboratory Guide, Vols 1-3, Cold Spring Harbor Press, the disclosure of which is incorporated herein by reference in its entirety.

Amino acid sequence insertions include amino- and/or carboxyl-terminal fusions ranging in length from one residue to polypeptides containing hundreds or more residues, as well as internal sequence insertions of one or more amino acid residues. As with any of the different variant types described herein, insertional variants are designed such that the resulting antibody possesses some physical, chemical and/or biological property not associated with the parental antibody from which it was derived. Methods for preparation of insertion variants are also routine and well known in the art (Sambrook et al., supra).

Fusion proteins comprising one or more of the chimeric scFv antibodies and another heterologous protein are a specific type of insertion variant contemplated by the invention. Examples of heterologous proteins which can be fused to a chimeric scFv antibody include proteins with long circulating half-life, such as, but not limited to, immunoglobulin constant regions; marker proteins; proteins or polypeptides that facilitate purification of the desired chimeric scFv antibody polypeptide; and polypeptide sequences that promote formation of multimeric proteins. Methods of making antibody fusion proteins are well known in the art. See, e.g., U.S. Pat. No. 6,306,393, the disclosure of which is incorporated herein by reference in its entirety. In certain aspects of the invention, fusion proteins are produced which may include a flexible linker, which connects the chimeric scFv antibody to the heterologous protein moiety. Appropriate linker sequences are those that do not affect the ability of the resulting fusion protein to be recognized and bind the epitope specifically bound by the V domain of the protein (see, e.g., WO 98/25965, the disclosure of which is incorporated herein by reference in its entirety).

Additionally, the chimeric scFv antibodies of the present invention can also be constructed to fold into multivalent V forms, which may improve binding affinity, specificity and/or increased half-life in blood. Multivalent forms of scFv antibodies can be prepared by techniques known in the art. One approach has been to link two scFv antibodies, such as two chimeric scFv antibodies of the invention, with linkers or disulfide bonds (Mallender and Voss, J. Biol. Chem. 269:199-2061994, WO 94/13806, and U.S. Pat. No. 5,989,830, the disclosures of which are incorporated herein by reference in their entireties). Another approach to making dimers of scFv antibodies is by adding sequences which are known to form a leucine zipper (Kostelny et al., J Immunol. 148(5): 1547-53 (1992); De Kruif et al., J. Biol. Chem. 271(13): 7630-34 (1996), the disclosures of which are incorporated by reference in their entireties). Another method is designed to make tetramers by adding a streptavidin-coding sequence at the C-terminus of the scFv. Streptavidin is composed of four subunits, so when the scFv-streptavidin is folded, four subunits associate to form a tetramer (Kipriyanov et al., Hum Antibodies Hybridomas 6(3): 93-101 (1995), the disclosure of which is incorporated herein by reference in its entirety). In yet another method, dimers, trimers, and tetramers are produced after a free cysteine is introduced in the parental protein. A peptide-based cross linker with variable numbers (two to four) of maleimide groups was used to cross link the protein of interest to the free cysteines (Cochran et al., Immunity 12(3): 241-50 (2000), the disclosure of which is incorporated herein in its entirety).

### III. Humanization

Humanized antibodies are also contemplated as an aspect of the invention. Humanized antibodies may be achieved by a variety of methods including, for example: (1) grafting the non-human CDRs onto a human framework and constant region (a process referred to in the art as humanizing through “CDR grafting”), or, alternatively, (2) transplanting the entire non-human variable domains, by “cloaking” them with a human-like surface by replacement of surface residues (a process referred to in the art as “veneering”). These methods are disclosed in, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991) the disclosures of each are incorporated herein by reference in their entireties. Humanization of mouse monoclonal antibodies by rational design has been reported in, for example, U.S. Patent Application Publication No. 20020091240 published Jul. 11, 2002, WO 92/11018 and U.S. Pat. No. 5,693,762, U.S. Pat. No. 5,766,866 (the disclosures of which are incorporated by reference herein in their entireties), and one of ordinary skill can readily utilize these techniques starting with a horse V domain or fragment thereof, or a complete chimeric scFv antibody, as described herein.

### IV. Other Modifications

The invention further contemplates additional modifications to one or more chimeric scFv antibodies in the plurality. In one aspect, the modifications are covalent in nature, and include for example, chemical bonding with one or more organic and/or inorganic moieties.

For example, the chimeric scFv antibodies are covalently modified to include one or more water soluble polymers, including polysaccharide polymers. Exemplary water soluble polymers include, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers. Such methods are known in the art, see, e.g. U.S. Pat. Nos. 4,640,835; 4,496,689; 4,301,144; 4,670,417; 4,791,192, 4,179,337, 4,766,106, 4,179,337, 4,495,285, 4,609,546 or EP 315 456, the disclosures of which are incorporated by reference in their entireties.

In one exemplary and non-limiting aspect, the water-soluble polymer is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is nontoxic, non-immunogenic, and approved by the Food and Drug Administration. Proteins or enzymes when conjugated to PEG have demonstrated bioactivity, non-antigenic properties, and decreased clearance rates when administered in animals.

Methods for preparing PEGylated chimeric scFv antibodies generally comprise the steps of (a) reacting the polypeptide with polyethylene glycol (such as a reactive ester or aldehyde derivative of PEG) under conditions whereby the polypeptide becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-pegylated product. In some embodiments, polypeptide will have a single PEG moiety at the N-terminus. See U.S. Pat. No. 5,234,784, herein incorporated by reference.

Chimeric scFv antibodies of the invention can also be conjugated directly to signal-generating compounds, e.g., by conjugation with an enzyme (see, e.g., Ngo et al., Mol. Cell. Biochem., 44:3-12, 1982; Maeda, M., J. Pharm. Biomed. Anal., 30:1725-1734, 2003, the disclosures of which are incorporated herein by reference in their entireties), fluorophore, and/or chemiluminescent compounds. Exemplary fluorophores and chemiluminescent compounds can be found in the Molecular Probes catalog (Molecular Probes, Inc., Eugene, Oreg.), and the references cited therein, all of which are incorporated herein by reference in their entireties. Procedures for accomplishing such labeling are well known in the art; for example, see Sternberger, L. A. et al., J. Histochem. Cytochem. 18:315 (1970); Bayer, E. A. et al., Meth. Enzym. 62:308 (1979); Engval, E. et al., Immunol. 109:129 (1972); Goding, J. W. J. Immunol. Meth. 13:215 (1976); and U.S. Pat. No. 4,391,904, the disclosures of which are incorporated herein by reference in their entireties.

### V. Purification of Chimeric ScFv Antibodies

In certain instances, it will be desirable to purify one or more of the chimeric scFv antibodies of the invention or variants thereof. Protein purification techniques are well known to those of skill in the art (see generally, Scopes, Protein Purification (Springer-Verlag, NY, 1982), the disclosure of which is incorporated herein in its entirety).

Generally, “purified” will refer to a composition comprising chimeric scFv antibodies that has been subjected to fractionation to remove various other components, and which composition substantially retains its expressed biological activity.

There is no general requirement that the chimeric scFv antibodies of the invention always be provided in its most purified state. Indeed, it is contemplated that less substantially purified chimeric scFv antibodies will have utility in certain embodiments. Partial purification may be accomplished by using fewer purification steps in combination, or by utilizing different forms of the same general purification scheme.

### VI. Binding Assays

The chimeric scFv antibodies of the invention may be screened for binding affinity to an antigen by methods well known in the art. Immunological binding assays typically utilize a capture agent to bind specifically to and often immobilize the analyte target antigen. The capture agent is a moiety that specifically binds to the analyte. Immunological binding assays are well known in the art [See, e.g., U.S. Pat. Nos. 4,366,241; 4,376,110; 4,517,288; and 4,837,168, Harlow and Lane, Antibodies, A Laboratory Manual, Ch 14, Cold Spring Harbor Laboratory, NY (1988), the disclosure of which are incorporated herein by reference in their entireties].

A. Non-Competitive Binding Assays:

Noncompetitive immunoassays can be used for diagnostic detection of an antigen in a sample. For example, a two-site, solid phase sandwich immunoassay may be used (Harlow and Lane, supra). In this type of assay, a binding agent, e.g., a chimeric scFv antibody, for an antigen is attached to a solid support. A second binding agent, which may also be an antibody, and which binds the antigen at a different site, is labeled. After binding at both sites on the antigen has occurred, the unbound labeled binding agent is removed and the amount of labeled binding agent bound to the solid phase is measured. The amount of labeled binding agent bound is directly proportional to the amount of antigen in the sample.

B. Competitive Binding Assays:

Competitive binding assays can be used for cross-reactivity determinations to permit a skilled technician to determine (1) if a protein or enzyme complex which is recognized by a chimeric scFv antibody of the invention is the desired protein and not a cross-reacting molecule or (2) to determine whether the antibody is specific for the antigen and does not bind unrelated antigens. Numerous types of competitive binding assays are well known in the art. See, e.g., U.S. Pat. Nos. 3,376,110, 4,016,043; Harlow and Lane, Antibodies: A Laboratory Manual, Cold Spring Harbor Publications, N.Y. (1988), all of which are incorporated herein by reference in their entireties.

C. Other Binding Assays:

Western blot methods are also valuable to detect or quantify the presence of antigen(s) in a sample (Hamada et al., J. Clin. Endocrinol. Metab. 61:120-128, 1985; Dennis-Sykes et al., J. Biol. Stand., 13:309-314, 1985, the disclosures of which are incorporated herein by reference in their entireties). The technique generally comprises separating sample proteins by gel electrophoresis on the basis of molecular weight and transferring the proteins to a suitable solid support, such as nitrocellulose filter, a nylon filter, or derivatized nylon filter. The sample is incubated with chimeric scFv antibodies or variants thereof that specifically bind the antigen and the resulting complex is detected. These antibodies may be directly labeled or alternatively may be subsequently detected using labeled antibodies that specifically bind to the antibody.

### VII. Therapeutic Uses

The present invention provides for both prophylactic and therapeutic methods of treating subjects (e.g., humans or other animals). In one aspect, the invention provides preventing or treating a disease or a disorder in a subject through prophylactic or therapeutic methods.

Administration of a therapeutic agent in a prophylactic method can occur prior to the manifestation of symptoms of an undesired disease or disorder, such that the disease or disorder is prevented or, alternatively, delayed in its progression. For example, short-term protection to a subject by passive immunization by the administration of one or more chimeric scFv antibodies of the invention, with or without adjuvants, is contemplated. Such passive immunization can be used for immediate protection of non-immunized individuals exposed to antigenic molecules that can result in an undesired disease or disorder.

As used herein, the terms “treating” or “treatment” includes the application or administration of a therapeutic agent to a subject who is afflicted with a disease, a symptom of disease or a predisposition toward an undesired disease or disorder, with the goal of curing, healing, alleviating, relieving, altering, remedying, ameliorating, improving or affecting the disease, the symptoms of disease or disorder or the predisposition toward the disease or disorder.

In another aspect, the invention contemplates the administration of a single chimeric scFv antibody, as well as combinations, or “cocktails,” of different antibodies. Such antibody cocktails may have certain advantages inasmuch as they contain antibodies which exploit different effector mechanisms. Such antibodies in combination can exhibit synergistic therapeutic effects. For example, two or more chimeric scFv antibodies from the plurality may be combined such that the combination of the antibodies together provide improved efficacy against a disorder to be treated. Compositions comprising one or more chimeric scFv antibodies may be administered to a subject already suffering from a disorder, or to a subject that may be in contact with antigenic molecules associated with a disorder to be treated.

A chimeric scFv antibody of the invention may be administered to a subject in need, by itself, or in a pharmaceutical composition where it is mixed with suitable carrier(s) or excipient(s) at doses to treat or ameliorate an undesired disease or disorder. Such a composition may also contain (in addition to a chimeric scFv antibody and a carrier) diluents, fillers, salts, buffers, stabilizers, solubilizers, and other materials well known in the art (Remington's Pharmaceutical Sciences 16th edition, Osol, A. Ed. (1980). The term “pharmaceutically acceptable” means a non-toxic material that does not interfere with the effectiveness of the biological activity of the active ingredient(s). The pharmaceutical composition may further contain other agents which either enhance the activity of the chimeric scFv antibody or compliment its activity or use in treatment. Such additional agents may be included in the pharmaceutical composition to produce a synergistic effect with a chimeric scFv antibody, or to minimize side effects. Techniques for formulation and administration of pharmaceutical compositions can be found in Remington's Pharmaceutical Sciences 16th edition, Osol, A. Ed. (1980), the disclosure of which is incorporated herein by reference.

Compositions comprising chimeric scFv antibodies can be administered for therapeutic and/or prophylactic treatments. As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such condition. When applied to an individual active ingredient, administered alone, the term refers to that ingredient alone. When applied to a combination, the term refers to combined amounts of the active ingredients that result in the therapeutic effect, whether administered in combination, serially or simultaneously.

Therapeutically effective amounts of a composition will vary and depend on the severity of the disease and the weight and general state of the subject being treated, but generally range from about 1.0 μg/kg to about 100 mg/kg body weight, or about 10 μg/kg to about 30 mg/kg, or about 0.1 mg/kg to about 10 mg/kg or about 1 mg/kg to about 10 mg/kg per application. Administration can be daily, on alternating days, weekly, twice a month, monthly or more or less frequently, as necessary depending on the response to the disorder or condition and the subject's tolerance of the therapy. Maintenance dosages over a longer period of time, such as 4, 5, 6, 7, 8, 10 or 12 weeks or longer may be needed until a desired suppression of disorder symptoms occurs, and dosages may be adjusted as necessary. The progress of this therapy is easily monitored by conventional techniques and assays.

In prophylactic applications, compositions comprising the chimeric scFv antibodies are administered to a subject not already in a disease state to enhance a subject's immune response to an antigen. Such an amount is defined to be a “prophylactically effective dose.” Again, effective amounts of a chimeric scFv antibody composition will vary and depend on the severity of the disease and the weight and general state of the subject being treated, but generally range from about 1.0 μg/kg to about 100 mg/kg body weight, or about 10 μg/kg to about 30 mg/kg, or from about 0.1 mg/kg to about 10 mg/kg or about 1 mg/kg to about 10 mg/kg per application. Typically, because a prophylactic dose is used in a subject prior to or at an earlier stage of disease, the prophylactically effective amount will be less than the therapeutically effective amount.

The exact dosage will be determined in light of factors related to the subject requiring treatment. Dosage and administration are adjusted to provide sufficient levels of the chimeric scFv antibody to maintain the desired effect. Specific dosages may be adjusted depending on conditions of disease, the age, body weight, general health conditions, sex, and diet of the subject, dose intervals, administration routes, excretion rate, combinations of drugs, and response to therapy. Any of the above dosage forms containing effective amounts are well within the bounds of routine experimentation and therefore, well within the scope of the instant invention.

The compositions of the present invention can be administered alone or as an adjunct therapy in conjunction with other therapeutics for the treatment of a disease or disorder. The effective amount of such other therapeutic agents depends on the amount of antibody present in the formulation, the type of disease, disorder, condition or treatment, and other factors discussed above.

Moreover, it is also contemplated that the methods of the present invention may be combined with other methods generally employed in the treatment of the particular disease or disorder that the subject exhibits. For example, in treatment of diseases or disorders for which decreasing chimeric scFv antibody levels ameliorates but does not eradicate the disorder, it may be advantageous to use additional compounds which eradicate the disorder. In other cases, it may be useful to administer drugs in addition to a chimeric scFv antibody in order to obtain additive or synergistic effects.

The frequency of dosing will depend upon the pharmacokinetic parameters of the pharmaceutical composition. Typically, a composition is administered until a dosage is reached that achieves the desired effect. The composition may therefore be administered as a single dose, or as multiple doses (at the same or different concentrations/dosages) over time, or as a continuous infusion. Long-acting pharmaceutical compositions may be administered every few days, every week, or every two weeks or every month or more depending on the half-life and clearance rate of the particular formulation. Further refinement of the appropriate dosage is routinely made. Appropriate dosages may be ascertained through use of appropriate dose-response data.

Pharmaceutical compositions of the invention can be administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the chimeric scFv antibody is suitably administered by pulse infusion, particularly with declining doses of the chimeric scFv antibody. In one aspect, the dosing is given by injections, either intravenous or subcutaneous, depending in part on whether the administration is brief or chronic. Other administration methods are contemplated, including topical, particularly transdermal, transmucosal, rectal, oral or by sustained release systems or by implantation devices. Where desired, the compositions may be administered by bolus injection or continuously by infusion, or by implantation device.

In a further aspect, the chimeric scFv antibodies of the invention can be administered to a subject that has been afflicted with environmental factors that evoke an immune response. Such environmental factors include atmospheric particulates, animal scratches, bites and stings, and the like. In another aspect, the chimeric scFv antibodies of the invention are immunospecific for a specific environmental factor. In one aspect, the plurality of chimeric scFv antibodies of the invention are biased towards immunospecific recognition of venomous extracts from one or more venomous animals.

Thus, pharmaceutical compositions comprising one or more of the chimeric scFv antibodies administered to humans or animals suffering from envenomation are specifically contemplated. In addition, secondary therapeutic agents may be administered in conjunction with the chimeric scFv antibodies of the invention to alleviate various other symptoms of envenomation. For example, scorpion venom contains several polypeptides which interfere with neuronal ionic balance and channel activity and generally manifests in the peripheral nervous system resulting in symptoms such as intense pain at the sting site lasting from minutes to twenty-four hours; swelling, itching, and a change in skin color; nausea and vomiting; anxiety, drowsiness, and fainting; increased saliva, tears, and sweat; numbness of the tongue; vision problems; diarrhea or inability to control bowels; swollen glands; altered heart activity; and paresthesia. Thus, it is an aspect of the invention to incorporate secondary therapeutic agents into a pharmaceutical composition to ameliorate these symptoms. Exemplary secondary therapeutic agents include, but are not limited to, local anesthetics to control paresthesia and pain at the sting site; antihistamines, steroids, hydrocortisone to reduce allergic reactions, swelling and itching; adrenergic blocking agents and vasodilators to counteract the scorpion-induced adrenergic cardiovascular effect; benzodiazepines to counteract scorpion-induced excessive motor activity and nervous system excitation; barbiturates to counteract scorpion-induced hyperactivity; anticholinergics to counteract scorpion-induced cholinergic symptoms; and vasopressors/inotropics to combat hypotension refractory to IV fluid therapy.

Other aspects, and advantages of the present invention will be understood upon consideration of the following illustrative examples, which are not intended to be limiting in any way.

### EXAMPLES

### Example 1

### Production of Horse Antibodies

The present Example describes the production of antibodies elicited in a horse that has been challenged with four different species of scorpions, namely Centruroides noxius, C. limpidus limipidus, C. limpidus tecomanus and C. suffusus suffusus. Immunization schemes, like those recommended in the literature, were followed with doses of venoms that ranged from 3 to 150 DL₅₀ per horse throughout twelve immunizations given over five to six weeks for the base schemes, and from 70 to 450 DL₅₀ per horse throughout five immunizations over three weeks for the reinforcement schemes, according to the type of venom applied. Freund's Complete and Incomplete adjuvants were used as well as a saline isotonic solution, using a total of 5, 10 or 20 ml in the different inoculations.

### Example 2

### Amplification of Horse Heavy Chain Variable Regions

Blood samples from a horse immunized as described in Example 1 were obtained from the Instituto Bioclon SA de CV. Lymphocytes were isolated by centrifugation over Lymphoprep (Gibco-BRL, Rockville, Md.) and used to extract total RNA as previously described (Chomczynski et al., Anal. Biochem., 162:156-159, 1987).

To amplify the horse immunoglobulin V_{H} fragments, total RNA isolated from the immunized horse was used for reverse transcription (RT) using the primer IGHG2-6REV: 5′-GTC CAC CTT GGT GCT GCTG-3′ (SEQ ID NO: 95), which corresponds to a conserved region of horse IGHC2-6 genes (Wagner et al., Immunogenetics, 54:353-364, 2002, the disclosure of which is incorporated herein by reference). The reaction was performed with the Protoscript® First Strand DNA Synthesis Kit (New England Biolabs).

Double-stranded DNA (dsDNA) was then obtained by PCR using the primers: HorVHForw1: 5′-CAG GTG CAR CTG MAG GAG TCR G-3′ (SEQ ID NO: 96) and HorJH5rev: 5′-GCC TCC ACC ACT CGA GAC GGT GAC CAG GAT ACC CTG-3′ (SEQ ID NO: 97). The underlined sequence in the latter primer corresponds to Xho I restriction site. The PCR reaction was performed in a total volume of 20 μL, containing dNTPs at a concentration of 2.5 mM, 4 μl of cDNA, 20 pmol of each primer, ThermoPol Reaction Buffer and 2 units of VentR® DNA Polymerase (New England Biolabs). The reaction mix was incubated at 94° C. for 3′, followed by 30 cycles of 1′ at 94° C., 1′ at 62° C., and 1′ at 72° C., and a final extension of 10′ at 72° C. The product obtained in this reaction was reamplified using primers HorJH5rev (described above) and HorVHFor1Sfi 5′-TTA CTC GCG GCC CAG CCG GCC ATG GCC CAG GTG CAR CTG MAG GAG TCR G-3′ (SEQ ID NO: 98) to add a Sfi I site (underlined), according to the procedure for obtaining dsDNA described above.

### Example 3

### Preparation of Phage Display Vector

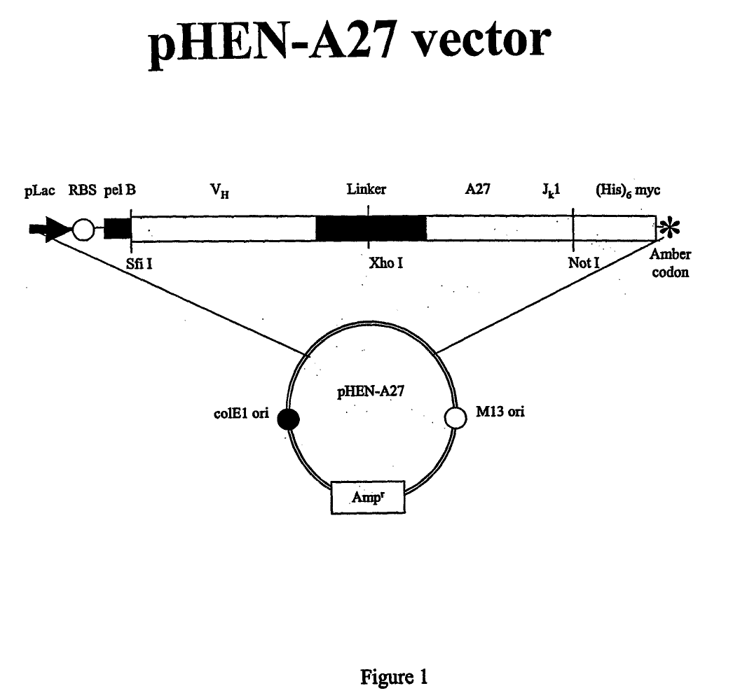

In order to display the chimeric scFv antibodies of the invention, a vector already encoding a human light chain variable region fragment (A27/Jk1) was produced (FIG. 1). The nucleotide (SEQ ID NO: 1) and amino acid (SEQ ID NO: 2) sequences of A27/Jk1 are set out below:

```
gaaattgtgttgacgcagtctccaggcaccctgtctttgtcaccagggga
E I V L T Q S P G T L S L S P G E
aagagccaccctctcctgcaggtccagccagagtgttttatacagctcca
  R A T L S C R S S Q S V L Y S S N
acaataagaactacttagcctggtaccagcagaaacctggccaggctccc
    N K N Y L A W Y Q Q K P G Q A P
aggctcctcatctatggtgcatccagcagggccactggcatcccagacag
R L L I Y G A S S R A T G I P D R
gttcagtggcagtgggtctgggacagacttcactctcaccatcagcagac
  F S G S G S G T D F T L T I S R L
tggagcctgaagattttgcagtgtattactgtcagcagtatggtagctca
    E P E D F A V Y Y C Q Q Y G S S
ccttggacgttcggccaagggaccaaggtggaaatcaaacgt
P W T F G Q G T K V E I K R
```

A27/Jk1 was synthesized by PCR in a single step reaction (Stemmer et al., Gene, 164:49-53, 1995, the disclosure of which is incorporated herein by reference in its entirety) using a set of overlapping oligonucleotides (Rojas et al. J Biotechnol. 94(3):287-98, 2002). The PCR reaction contained dNTPs (New England Biolabs) at a concentration of 2.5 mM, 1 pmol of each internal primer, 40 pmol of the amplification primers, ThermoPol Reaction Buffer and 2 unit of VentR® DNA Polymerase (New England Biolabs) in a final volume of 20 μL. The PCR mix was initially incubated at 94° C. for 3′, followed by 30 cycles of 1′ at 94° C., 1′ at 65° C., and 1′ at 72° C., and a final extension of 10′ at 72° C. The amplicon obtained in this reaction was gel-purified in a Tris-Borate 2.5% agarose gel with the Gel Extraction Kit (QIAquick from QIAGEN). The product was double digested with Xho I and Not I (New England Biolabs) at a ratio of 20 enzyme units/μg of DNA and cloned in a derivative of the pHEN-1 vector (Hoogenboom et al., Nucleic Acids, Res., 19:4133-4137, 1991) to yield the pHEN-A27 vector.

### Example 4

### Cloning and Display in Phase of the Plurality of Chimeric scFv Antibodies

V_{H} fragments were gel-purified with the Gel Extraction Kit (QIAquick from QIAGEN) and sequentially digested with Xho I and Sfi I (New England Biolabs) at a ratio of 20 enzyme units/μg of DNA. The digested fragments were ligated into 1 μg of the pHEN-A27 vector (FIG. 1) at a molar ratio of 1:6 (vector: insert) with the Quick Ligation Kit (New England Biolabs). The ligation mix was purified and concentrated using QIAquick and then electroporated in TG1 electrocompetent cells (Stratagene) to yield a plurality of 2.3×10⁸ transformed units (tu).

Transformed cells were grown overnight in 2×TY-agar plates containing 100 μg/mL carbenicillin and 1% glucose. The plates were scraped with 10 mL of 2×TY. 50 μL of cells suspension were used to inoculate 50 mL of 2×TY containing 100 μg/mL carbenicillin and 1% glucose, grown until the OD at 600 nm reached 0.4 units and infected with KM13 helper phage (Goletz et al. J Mol. Biol. 315(5):1087-97, 2002; the disclosure of which is incorporated herein by reference). The infected culture was grown overnight in 2×TY containing 100 μg/mL carbenicillin, 50 μg/mL kanamycin and 1% glucose, centrifuged and phages were PEG-purified. The plurality of chimeric scFv antibodies was titrated and stored in 15% glycerol at −80° C. until used.

### Example 5

### Sequencing of the Horse V_{H} Fragments

Forty-seven clones randomly selected from the plurality prior to antigenic selection were grown for 8 hours in 5 mL of 2×TY containing 100 μg/mL carbenicillin and 1% glucose. The cultures were then centrifuged and the plasmid DNA purified using the Miniprep Kit Spin QIAprep® from QIAGEN. Sequencing was performed using the ABI Prism Big Dye Terminator Cycle Sequencing Kit v3.1. The sequencing reaction was performed in a total volume of 10 μL containing 2 μL of Big Dye mix, 1× of sequencing buffer, 3.56 pmoles of sequencing primer and 400 ng of purified plasmid. Reactions were run following the ABI Prism protocol specifications.

Forty-six clones (SEQ ID NOs: 49-94) were found to be unique at the amino acid level and are aligned below in Table 2.

TABLE 2
Alignment of Horse V_{H} Sequences

| | \|... \|....\|.... \|....\|.... \|....\| .ab...\|....\|.... \|.... \|..abcdefghi..\|.... |
|---|---|
| 1-2: | QVQLKESGPGLVKPSQTLSLTCTVSGLSVSS--NGVAWVRQAPGKGLEFVGVIH---------TDGGVDY |
| 1-3: | ..........................F.LNT--YA.G..............S.Y---------SI.SAT. |
| 1-4: | ..........................S.SEG--Y..G.......R......G.T---------NS.SARF |
| 1-5: | ....Q.......................L..--.T.G...........Y..A.Y---------GSASAA. |
| 1-7: | ............................DN.--.A.G.............ADLT------------DSAS |
| 1-10: | .............S............A.LND--IA.G...........Y..CVY---------DGT.EN. |
| 1-11: | ..........................F.LIT--DS.G..............GLS---------SF-SAN. |
| 1-13: | ....Q.......M...............LVNR-.A.R...........Y..S.Y---------GVEERN. |
| 1-14: | ............................L..--.T.G...........Y..I.Y---------GSASTL. |
| 2-1: | ......................S....NLNE--DI.G.........P.Y..S.W---------G.RSPK. |
| 2-3: | .......................I....L..--Y..G..............G.R---------SS.SAN. |
| 2-4: | ............................L..--VD.G...........Y.SW.G-----------RSTS. |
| 2-5: | ..............I.............L..--.D.G...........Y.AR.W---------GGANEH. |
| 2-6: | ...........................LLN.--.C.G........R..Y..S.Y----------GTLTN. |
| 2-7: | ............................LTG--SQI............YISGSS--------------M. |
| 2-8: | ............................LTD--Y..G.............AR.D---------S..SKNF |
| 2-9: | ....................V.....F.LTD--R..G.............SY.L---------.S.AQ.G |
| 2-11: | .........D.M..............F.L..--Y..G..............GLP---------GS.SA.. |
| 2-14: | ............................L..--Y..D...........W..G.T---------SS..SG. |
| 2-15: | ....Q.................S.....L..--VF.Y...........Y..F.GNSGSTIGNSGKTNYN. |
| 3-1: | ....................I.....F.L..--DS.G...............V-----------SS.RAR |
| 3-2: | .........D.....E....V.S...Q.L..--YD.G.......W.......TA---------HY..I.. |
| 3-3: | ............................L..--Y.AG....S......Y..GVG---------KS.SSN. |
| 3-4: | ............................LRG--.V.G...........H..ENV---------SS..AF. |
| 3-5: | ......................A...F.L..--D.IN..............S.Y---------.SASTI. |
| 3-6: | ....Q.......R.AE...........DL..--GTII...........R..E.V---------GE.SGF. |
| 3-7: | ............................L..--SC.Q....V......Y..R.V--------SSG..LT. |
| 3-8: | ..............A...T........HLN.D-AV.G..............GLS---------NT.RAN. |
| 3-9: | ................A......I..F.LT.--H..G..............S.W---------.T.QTIN |
| 3-12: | ....................V.....F.LN.--W..G...........E..GSQ---------IG.NAN. |
| 3-13: | ..............G.....S.......L.T--.T.G.........W.Y.AALY---------A.ADG.. |
| 3-15: | ............N.........F...F.LT.--WH.G..............G.P---------VI.EAY. |
| 4-1: | ....1.......................LN.--YD.N...........V..S.S---------DS.IAV. |
| 4-2: | ......................A...F.LRD--AAMG.......R...YI.SMY-----------IRE.. |
| 4-3: | ..........................F.L.T--T..G..............GVP---------SS.SAN. |
| 4-4: | .......................I..F.LT.--AS.D..............G.A---------.S.RAN. |
| 4-5: | ..........................M.L.T--.T.G...........Y..L.Y---------GMKSAE. |
| 4-6: | ....Q.....................I.LTD--YN.D..............GLW---------.N.QSN. |
| 4-7: | ..........................NDLR.--F.................GVA---------RF.SPY. |
| 4-8: | .........................A..L..--A..G.............AG.V---------GD..TYA |
| 4-9: | ....Q.......................L..--.A.G...........Y.DS.G---------NSESANF |
| 4-10: | ..........................FPL..--Y..G...........S..E.A---------SS.SAN. |
| 4-11: | ....Q.......................L..--.VLG...........WI.G.Y---------GSASPN. |
| 4-12: | ............................L..--Y..G..............G.L---------SS.RAN. |
| 4-14: | ......................A....PLRD--AA.G...........YI.SMY-----------NEE.. |
| 4-15: | ....Q.....Q.............T.G.ITNKYSSWT.L..P.......I.Y.Y---------Y..RRY. |

| | \|.... \|....\|.... \|.... \|..abc..\|....\|.... \|.... \|abcdefghijklmno....\|.. |
|---|---|
| 1-2: | NPALKSRGSITRDISKSQLYLTLNTLTGEDTAVYYCARHASTGA---------YLYPFDYWGQGIL |
| 1-3: | .LD....V...K.T....V...V.S..S........G.RVNEI---------------........ |
| 1-4: | ..G.A..A..LKNTE...V....TD............KDSES.F------LYWGH.GVE....... |
| 1-5: | .......A...K.T....V.....S..S.........GGGGGWI----GYDYLGY.DIN....... |
| 1-7: | .......VR..KEP....VR.IM.S..E........IHGYYNSF---------MVGAIK....... |
| 1-10: | .......A.....T....V..A..S..S........TGGKGDYG---RYWNSYAEDGITN...... |
| 1-11: | ..G.N..A...K.T..G.VV....S..SD.......VSFSGQ.E---AFAFAYLY.GIT....... |
| 1-13: | ..V....VD..K.T....V.....SV.SG.........NEYGI---------------VE...... |
| 1-14: | .......A...KES....V.....S..S.........GGF.GFD--------WFDRGIN....... |
| 2-1: | ..DV...A..SK.T..R.V..Q..S.SD.........GGLTILG------VMKDETFV.H..P... |
| 2-3: | .......A...K.T.Q.HV.....S..S.........GGTEQRD--------YIDVGVKF...... |
| 2-4: | K......A...K.T....A.....S..S........VGGYAD.I--------------........ |
| 2-5: | .......A...K.T....V.....S..S........GGTPGFYN--------SAYET.A....... |
| 2-6: | .S..R..AR..S.Y....VL....S..S.........ALDYGVT---------ISRDIND...... |
| 2-7: | .....F.A...K.T..N.VT....K...........VAT.FW.G----------YGGIQ....... |
| 2-8: | .......AN.IK.T....V.....S..S.........GYGYS.R-------YSTPGNLYW...... |
| 2-9: | ....R..V.....T.L..V...M.SV..........G..GPNLH-----------GT......... |
| 2-11: | S...R..A...K.T....V.V...S..S..........FYNWNS------GVVSYTGI........ |
| 2-14: | .......A...K.T....V.....S..S.........GEEEGYV-----YGFTRY.GNY....... |
| 2-15: | ..V....A..SK.T....VL....S..S.........GDNI-----------------K....... |
| 3-1: | .......A...K.T.E..V.....S..S.........GGR.GYS-----YYAGMVDGIN....... |
| 3-2: | .......A...K.T..N..T.I..S..S........TGE.Q.NC-------DFGVSCLG....... |
| 3-3: | .S...P.A...K.S....IS...RS............IYD.YLR----------GWSVV........ |
| 3-4: | S......A.....T....I.....S..R.........AWKVSSR--------S..DGIN........ |
| 3-5: | .......A...K.T....V.....S..S........SGGSE-----------------E........ |
| 3-6: | .......AM..K.T..NEI....KS..S.........GAWGGNY-----YENFFINGVEN....... |
| 3-7: | .......A.....T....V.....S..D........TGALN.HY------SSYAG.GI......... |
| 3-8: | .......AI..K.T....V.....S..S....D.F..GGRMFDY------VYGGY.EIQ........ |
| 3-9: | ..T....V.....TGLN.VS....E..S.........GG.ISDYDFFGFRGMFSI.DVQ........ |
| 3-12: | ....E..A...K.A....V.....S..E........TGGYNWNL-------GTNRDRIT........ |
| 3-13: | ..V.Q..A...K.T..N.VF...D...S........TGGVFSVP--VGTGYTY.ESGIL........ |
| 3-15: | ..V....I...K.T....V.....S..D......A...LRNWYG--------D.YSDM......... |
| 4-1: | .......A...K.T.N..V.....S.............GNFAF---------------......... |
| 4-2: | .......A.V.K.TKE.RS.....A..S......W.VGDVG..---------------Y........ |
| 4-3: | .......C...K.E....V.....S..S......I..GGFYNTL----------DKGIN........ |
| 4-4: | ..V....AT....T....V.....S............ESYYD.V----------GGNYYF....... |
| 4-5: | .......A...K.T.N..VL....S..S.........GGEAW.P---MYSSNEEKNGVE........ |
| 4-6: | .......AR..K.T....V.....S..S........EGYGNSWQ------------.PH........ |
| 4-7: | .......AI..K.T..KESV....SV........W..GGYGDES-------------WGP....... |
| 4-8: | ....R..A...K.T....V.....M..S.........GSLEFSG------WGVMR.GIN........ |
| 4-9: | .......A...E.T...RV.....S..S.........AQYDYF.----GAYGLIP.AIK........ |
| 4-10: | .......A...K.T....V.....S..S........TGWGLRL---------------Y........ |
| 4-11: | .LT..A.....K.T....V....TGM.E.........GG.PYNY------AGGNIGRMK........ |
| 4-12: | .......A.....TT.N.V.....S......S......SFAS.G-------SY.D.AINF....... |
| 4-14: | ..D....A.V.K.T...RVT....S..S........VGDGGS.---------------Y........ |
| 4-15: | ..SF...T..S..T.RNEFS.Q.SSV.D..A...F..GDYGY.G-------WV.SDGEN........ |

The nucleotide sequence of clone 2-1 was found twice and its translation product is therefore included only once in the alignment. All amino acid sequences have a pattern compatible with that of functional V_{H} domains (Chothia et al., J. Mol. Biol., 196:901-907, 1987; Almagro et al., Mol. Immunol., 34:1199-1214, 1997, the disclosures of which are incorporated herein by reference in their entireties). The resulting nucleotide sequences of the forty-six unique clones are available at GenBank (accession numbers: DQ125413-DQ125458) and are set out below in Table 3.

TABLE 3
Horse V_{H} Nucleotide Sequences

| | | | |
|---|---|---|---|
| | Genbank | | SEQ |
| | Accession | | ID |
| Clone | No. | Nucleotide Sequence | NO: |

| | | | | |
|---|---|---|---|---|
| 1-2 | DQ125413 | 1 | caggtgcaac tgaaggagtc aggacctggc ctggtgaagc cctagcagac cctctccctc | 3 |
| | | 61 | acctgcactg tctctggatt atcagtgagc agtaatggtg tggcctgggt ccgccaggct | |
| | | 121 | ccaggaaaag ggctggaatt tgtcggtgtt atacatactg atggaggtgt tgactacaac | |
| | | 181 | ccagccctga agtcccgagg cagcatcact agggacatct caaagagcca actttatctg | |
| | | 241 | acgctgaaca cactgacagg cgaggacacg gccgtctatt actgtgcgcg acatgctagt | |
| | | 301 | actggtgctt acctttaccc ctttgactat tggggccagg gtatcctggt caccgtctcg | |
| 1-3 | DQ125414 | 1 | caggtgcaac tgaaggagtc gggacctggc ctggtgaagc cctcgcagac cctctccctc | 4 |
| | | 61 | acctgcactg tctctggatt ctctttgaac acttacgcag tgggatgggt ccgccaggct | |
| | | 121 | ccaggaaaag gcctggaatt tgttggtagt atttatagta ttggaagtgc gacgtacaat | |
| | | 181 | ttagacctga agtcccgagt cagcatcacc aaggacacct caaagagcca agtttatctg | |
| | | 241 | acggtgaata gtctgacaag tgaggacacg gccgtctatt attgtggaag acgagtcaat | |
| | | 301 | gaaattgact actggggcca gggtatcctg gtcaccgtct cg | |
| 1-4 | DQ125415 | 1 | caggtgcaac tgaaggagtc aggacctggc ctggtgaagc cctcgcagac cctctccctc | 5 |
| | | 61 | acctgcactg tctctgggtc atcttcggag ggttatggtg tgggctgggt ccgccaggct | |
| | | 121 | ccaggacgag gactagagtt tgtagggggt ataaccaata gtggtagtgc aagatttaat | |
| | | 181 | ccaggactgg cgtcccgagc cagcattctc aagaacaccg aaaagagcca agtttacctg | |
| | | 241 | acgctgaccg acctgacagg cgaggacacg gccgtctatt attgtgcgaa ggattccgag | |
| | | 301 | agtggctttc tttattgggg acattacggt gtagaatatt ggggccaggg tatcctggtc | |
| | | 361 | accgtctcg | |
| 1-5 | DQ125416 | 1 | caggtgcagc tgcaggagtc aggacctggc ctggtgaagc cctcgcagac cctctccctc | 6 |
| | | 61 | acctgcactg tctctgggtt atctttgagc agtaatactg taggctgggt ccgccaggct | |
| | | 121 | ccaggaaaag gactggaata cgttggtgct atatatggta gtgcaagtgc agcgtacaac | |
| | | 181 | ccagccctga agtcccgagc cagcatcacc aaggacacct caaagagcca agtttatctg | |
| | | 241 | acgctgaaca gcctgacaag cgaggacacg gccgtctatt actgtgcagg aggaggcggt | |
| | | 301 | ggttggattg gttatgacta cttaggatat tatgatataa actactgggg ccagggtatc | |
| | | 361 | ctggtcaccg tctcg | |
| 1-7 | DQ125417 | 1 | caggtgcagc tgaaggagtc gggacctggc ctagtgaagc cctcgcagac cctctccctc | 7 |
| | | 61 | acctgcactg tctctggatt atctgacaac agtaacgctg tgggctgggt ccgccaggct | |
| | | 121 | ccaggaaaag gactggaatt tgtggctgat ctaacggata gtgacagtaa cccagccctg | |
| | | 181 | aagtcgcgag tcaggatcac caaggaaccc tcaaagagcc aagttcgcct gattatgaac | |
| | | 241 | agcctgacag aagaggacac ggccgtctat tactgtattc atggttacta caatagtttt | |
| | | 301 | atggtgggag cgataaaata ttggggccag ggtatcctgg tcaccgtctc g | |
| 1-10 | DQ125418 | 1 | caggtgcaac tgaaggagtc aggacctggc ctggtgaagt cctcgcagac cctctccctc | 8 |
| | | 61 | acctgtactg tctctggggc gtccttgaac gacattgctg tgggttgggt ccgccaggct | |
| | | 121 | ccaggaaaag gactggaata cgttggttgt gtttatgatg gtaccggaga aaactataac | |
| | | 181 | ccagccctga agtcccgagc cagcatcacc agggacacct caaagagcca ggtttatctg | |
| | | 241 | gcgctgaaca gcttgacgag tgaggacacg gccgtctatt attgtacagg aggcaagggt | |
| | | 301 | gactatggta gatactggaa tagttacgct gaggatggaa taaccaactg gggccagggt | |
| | | 361 | atcctggtca ccgtctcg | |
| 1-11 | DQ125419 | 1 | caggtgcaac tgaaggagtc aggacctggc ctggtgaagc cctcgcagac cctgtccctc | 9 |
| | | 61 | acttgcactg tctctggatt ctctttgatc actgacagtg taggctgggt ccgccaggct | |
| | | 121 | ccagggaaag ggctggaatt tgttggtgga ctttctagtt ttggaagtgc aaattacaac | |
| | | 181 | ccaggcctga actcccgagc cagcatcacc aaggacacct caaagggcca agtcgttctg | |
| | | 241 | acgctgaaca gcctgacaag cgacgacacg gccgtctatt actgtgtgtc attttcgggc | |
| | | 301 | cagggtgaag cgttcgcttt cgcttacctt tattatggaa taacctactg gggccagggt | |
| | | 361 | atcctggtca ccgtctcg | |
| 1-13 | DQ125420 | 1 | caggtgcaac tgcaggagtc aggacctggc ctggtgatgc cctcgcagac cctctccctc | 10 |
| | | 61 | acctgcactg tctctggatt atctctggtg aacaggaatg ctgtgcgctg ggtccgccag | |
| | | 121 | gctccgggaa aagggctgga atacgttggt tcaatatacg gtgttgaaga acgaaactac | |
| | | 181 | aacccagtcc tgaagtcccg agtagatatc accaaggaca cctcaaagag tcaagtttat | |
| | | 241 | ctgacgctga atagcgtgac aagcggggac acggccgtct attactgtgc gagaaatgaa | |
| | | 301 | tatggtattg tggaatgggg ccagggtatc ctggtcaccg tctcg | |
| 1-14 | DQ125421 | 1 | caggtgcagc tgaaggagtc aggacctggc ctggtgaagc cctcgcagac cctctccctc | 11 |
| | | 61 | acctgcactg tctctggatt atctttgagc agtaatactg tagggtgggt ccgccaggct | |
| | | 121 | ccaggaaaag ggctggagta cgtcggtatt atctatggta gtgcaagtac attgtacaac | |
| | | 181 | caagccctga agtcccgagc cagcatcacc aaggaatcct caaagagcca agtttatttg | |
| | | 241 | acgctgaaca gcctgacaag cgaggacacg gccgtctatt attgtgcagg aggctttagc | |
| | | 301 | ggctttgatt ggttcgatag aggtataaac tactggggcc agggtatcct ggtcaccgtc | |
| | | 361 | tcg | |
| 2-1 | DQ125422 | 1 | caggtgcaac tgaaggagtc gggacctggc ctggtgaagc cctcgcagac cctcagcctg | 12 |
| | | 61 | acatgcagtg tctctggatt gaatttgaac gaagatattg tagggtgggt ccgccaggct | |
| | | 121 | ccaggaaaag ggccggaata cgtcggaagt atatggggag atagaagccc aaaatacaat | |
| | | 181 | ccagacgtga agtcccgagc cagtatcagt aaggacacct cgaaacgcca ggtttatctt | |
| | | 241 | caactgaaca gcctgagtga cgaggacacg gccgtctatt actgtgcagg aggacttaca | |
| | | 301 | attttaggcg tcatgaagga tgagacgttc gtggatcact ggggcccggg tatcctggtc | |
| | | 361 | accgtctcg | |
| 2-3 | DQ125423 | 1 | caggtgcagc tgaaggagtc aggacctggc ctggtgaagc cctcgcagac cctctccctc | 13 |
| | | 61 | acctgcacta tctctggatt atctttgagc agctatggtg tgggctgggt ccgccaggct | |
| | | 121 | ccaggaaaag ggctggaatt cgttggtgga atacgtagta gtggaagtgc aaactacaat | |
| | | 181 | ccagccctga agtcccgagc cagcatcacc aaggacacct cacagagcca tgtttatctg | |
| | | 241 | acgctgaaca gcctgacaag cgaggacacg gccgtctatt actgtgcagg agggacagaa | |
| | | 301 | caacgtgatt atattgacgt tggtgtgaag ttctggggcc agggtatcct ggtcaccgtc | |
| | | 361 | tcg | |
| 2-4 | DQ125424 | 1 | caggtgcaac tgaaggagtc aggacctggc ctggtgaagc cctcgcagac cctctccctc | 14 |
| | | 61 | acctgcactg tctctggatt atctttgagc agtgttgatg taggctgggt ccgccaggct | |
| | | 121 | ccaggaaaag gactggaata cgttagttgg ataggtagaa gtactagcta caagccggcc | |
| | | 181 | ctgaagtccc gagccagcat caccaaggac acctcaaaga gccaagctta tctgacgctg | |
| | | 241 | aacagtctga cgagcgagga cacggccgtc tattactgtg taggaggtta cgcggacggt | |
| | | 301 | atagattact ggggccaggg tatcctggtc accgtctcg | |
| 2-5 | DQ125425 | 1 | caggtgcagc tgaaggagtc aggacctggc ctggtgaaga tctcgcagac cctctccctc | 15 |
| | | 61 | acctgcactg tgtctggatt atctttgagc agtaatgatg taggctgggt ccgccaggct | |
| | | 121 | ccaggaaaag ggctggaata cgtggctcgt atatggggtg gtgcaaatga acactacaac | |
| | | 181 | ccagccctga agtcgcgagc cagcatcacc aaggacacct caaagagcca agtttatctg | |
| | | 241 | acgctgaaca gcctgacaag cgaggacacg gccgtctatt actgtggagg aacacctggt | |
| | | 301 | ttctataata gtgcttacga gacgtttgcc tactggggcc agggtatcct ggtcaccgtc | |
| | | 361 | tcg | |
| 2-6 | DQ125426 | 1 | caggtgcaac tgaaggagtc aggacctggc ctggtgaagc cctcgcagac cctttccctc | 16 |
| | | 61 | acctgcactg tctctggatt acttttgaac agtaattgtg taggttgggt ccgccaggct | |
| | | 121 | ccaggaaaac gactggaata cgttggttct atatatggga cgttaacaaa ctacaactca | |
| | | 181 | gccctgaggt cccgagccag aatcaccagc gactactcaa agagccaagt tcttctgacg | |
| | | 241 | ctgaacagcc tgacaagcga ggatacggcc gtctattact gtgcagcact cgattatggt | |
| | | 301 | gtgacgatta gtcgcgatat aaatgattgg ggccagggta tcctggtcac cgtctcg | |
| 2-7 | DQ125427 | 1 | caggtgcagc tgaaggagtc aggacctggc ctggtgaagc cctcgcagac cctctccctc | 17 |
| | | 61 | acctgcactg tctctggatt gtctttgaca ggtagtcaga tagcttgggt ccgccaggct | |
| | | 121 | ccaggaaaag gactggaata tattagtgga agttcaatgt acaacccagc cctgaagttc | |
| | | 181 | cgagccagca tcaccaagga cacctccaag aatcaagtta ctctgacgct gaataagctg | |
| | | 241 | acaggcgagg acacggccgt ctattactgt gtggcgacag ctttttgggg cggttatggc | |
| | | 301 | ggtatccaat actggggcca gggtatcctg gtcaccgtct cg | |
| 2-8 | DQ125428 | 1 | caggtgcagc tgaaggagtc aggacctggc ctggtgaagc cctcgcagac cctctccctc | 18 |
| | | 61 | acctgcactg tctctggatt atctttgaca gattatggtg tgggctgggt ccgccaggct | |
| | | 121 | ccaggaaaag ggctggaatt tgttgccaga atagatagtg atggaagtaa aaactttaac | |
| | | 181 | ccagcgctga agtcccgagc caacatcatc aaggacacct caaagagcca agtttatctg | |
| | | 241 | acgctgaaca gcctgacaag tgaagacacg gccgtctatt actgtgcagg gtatggttac | |
| | | 301 | agtggtcgtt actccacacc ggggaattta tactggtggg gccagggtat cctggtcacc | |
| | | 361 | gtctcg | |
| 2-9 | DQ125429 | 1 | caggtgcagc tgaaggagtc gggacctggc ctagtgaagc cctcgcagac cctgtccctc | 19 |
| | | 61 | gtctgcactg tcagtggatt ctccctgacc gaccggggtg taggctgggt ccgccaggcg | |
| | | 121 | ccaggaaaag gactggaatt tgtgagttat atactaacca gtggagccca agacgggaat | |
| | | 181 | ccagccctaa ggtcccgagt cagcatcacc agggacacct cactgagtca agtttatctg | |
| | | 241 | acaatgaaca gcgtgacagg cgaggacacg gccgtctact attgtgggag gcatggaccg | |
| | | 301 | aatcttcatg gaacttttga ctattggggc cagggtatcc tggtcaccgt ctcg | |
| 2-11 | DQ125430 | 1 | caggtgcagc tgaaggagtc aggacctgac ctgatgaagc cctcgcagac cctctccctc | 20 |
| | | 61 | acctgcactg tctctggatt ctctttgagc agttatggtg taggctgggt ccgccaggct | |
| | | 121 | ccaggtaaag gcctggagtt tgttggcggg ttacctggta gtggaagtgc agactacagc | |
| | | 181 | ccagccctga ggtcccgagc cagcatcacc aaggacacct caaagagcca agtttatgtg | |
| | | 241 | acgctgaaca gcctgacaag cgaggacacg gccgtctatt actgtgcaag attctataac | |
| | | 301 | tggaatagtg gtgttgtcag ttatactggt attgactact ggggccaggg tatcctggtc | |
| | | 361 | accgtctcg | |
| 2-14 | DQ125431 | 1 | caggtgcaac tgaaggagtc aggacctggc ctggtgaagc cctcgcagac cctctccctc | 21 |
| | | 61 | acctgcactg tctctggatt atctttgagc agttatggtg tggactgggt ccgccaggct | |
| | | 121 | ccaggaaaag gacttgaatg ggttggtggt ataactagta gtggaggttc aggttacaac | |
| | | 181 | ccagccctga agtcccgagc cagcatcacc aaggacacct caaagagcca agtttatctg | |
| | | 241 | acgctgaaca gcctgacaag cgaggacacg gccgtctatt aatgtgcagg agaggaggaa | |
| | | 301 | ggctacgttt atggttttac tcgttattat gntaactact actggggcca gggtatcatg | |
| | | 361 | gtcaccgtct cg | |
| 2-15 | DQ125432 | 1 | caggtgcagc tgcaggagtc aggacctggc ctggtgaagc cctcgcagac cctctccctc | 22 |
| | | 61 | acctgcagtg tctctggatt gtctttgagc agtgtttttg tatactgggt ccgccaggct | |
| | | 121 | ccaggaaaag ggctggaata tgttggtttt ataggtaata gtggaagtac aataggtaat | |
| | | 181 | agtggaaaaa caaactacaa ctacaaccca gtcctgaagt cccgagccag catcagcaag | |
| | | 241 | gacacctcaa agagccaagt tcttctgacg ctgaacagcc tgacaagcga ggacacggcc | |
| | | 301 | gtctattact gtgcaggaga caatataaag tattggggcc agggtatcct ggtcaccgtc | |
| | | 361 | tcg | |
| 3-1 | DQ125433 | 1 | caggtgcaac tgaaggagtc aggacctggc ctggtgaagc catcgcagac cctctccctc | 23 |
| | | 61 | atctgcactg tctctggatt ctctttgagc agtgacagtg taggctgggt ccgccaggct | |
| | | 121 | ccaggaaaag ggctggaatt tgttggagtg gtacatagta gtggaagggc aagaaaccca | |
| | | 181 | gccctgaagt cccgagccag catcaccaag gacacctcag agagccaagt ttatctgacg | |
| | | 241 | ctgaacagcc tgacaagcga ggacacggcc gtctattact gtgcaggggg gcgtagtggc | |
| | | 301 | tacagttatt acgctgggat ggtagatggt ataaactact ggggccaggg tatcctggtc | |
| | | 361 | accgtctcg | |
| 3-2 | DQ125434 | 1 | caggtgcaac tgaaggagtc cggacctgac ctggtgaagc cataggagac cctctccctc | 24 |
| | | 61 | gtctgctccg tctctggaca atctttgagc agttatgatg tgggctgggt tcgccaggct | |
| | | 121 | ccaggctggg gactggaatt cgttggtgta acggcgcatt atggaggtat agactacaat | |
| | | 181 | ccagccctga agtcccgagc cagcatcacc aaggacacct caaagaacca acttactctg | |
| | | 241 | atactgaata gtctgacaag cgaggacacg gccgtctatt actgtacagg agaagcgcag | |
| | | 301 | actaattgtg actttggcgt cagttgtttg ggctactggg gccagggtat cctggtcacc | |
| | | 361 | gtctcg | |
| 3-3 | DQ125435 | 1 | caggtgcaac tgaaggagtc gggaccgggc ctggtgaagc cctcgcagac cctctccctc | 25 |
| | | 61 | acctgcactg tctctggatt aagtttgagc agttatggtg caggctgggt ccgccagtct | |
| | | 121 | ccaggaaaag ggctggaata tgttggtggg gtgggtaaaa gtggaagttc aaattacaat | |
| | | 181 | tcagccctga agccccgagc cagtatcacc aaggactcct caaagagtca gatttctctg | |
| | | 241 | acgctgagaa gcctgacagg cgaggacacg gccgtctatt actgtgcgat ctacgatagt | |
| | | 301 | tatcttcgtg gttggtcagt tgtctactgg ggccagggta tcctggtcac cgtctcg | |
| 3-4 | DQ125436 | 1 | caggtgcagc tgaaggagtc aggacctggc ctggtgaagc cctagcagac cctctccctc | 26 |
| | | 61 | acctgcactg tctctggatt atctttgaga ggtaatgttg taggctgggt ccgccaggct | |
| | | 121 | ccaggaaaag ggctggaaca cgttggcgaa aacgttagta gtggaggtgc gttctacagc | |
| | | 181 | ccagccctaa agtcccgagc cagcatcacc agggacacct caaagagcca aatttatctg | |
| | | 241 | acgctgaaca gcctgacaag ggaggacacg gccgtctatt actgtgcagc atggaaggtt | |
| | | 301 | agcagtcgct cttacttgga tggtataaac tactggggcc agggtatcct ggtcaccgtc | |
| | | 361 | tcg | |
| 3-5 | DQ125437 | 1 | caggtgcaac tgaaggagtc aggacctggc ctggtgaagc cctcgcagac cctctccctc | 27 |
| | | 61 | acctgcgctg tctctggatt ctctttgagc agtgacggta taaactgggt ccgccaggct | |
| | | 121 | ccaggaaaag ggctggaatt cgtgggttct atatatacta gtgcaagtac aatctacaac | |
| | | 181 | ccagccctga agtcccgagc cagcatcacc aaggacacct caaagagcca agtttatctg | |
| | | 241 | acgctgaaca gcctgacaag tgaggacacg gccgtctatt actgttcagg aggcagtgaa | |
| | | 301 | gaatattggg gccagggtat cctggtcacc gtctcg | |
| 3-6 | DQ125438 | 1 | caggtgcaac tgcaggagtc aggtcctggc ctggtgaggc cagcagagac cctctccctc | 28 |
| | | 61 | acctgcactg tctctggatt ggacttgagc agtggtacga taatctgggt ccgccaggct | |
| | | 121 | ccaggaaaag ggctggagag agtcggtgaa atagttggtg agggaagtgg attctacaat | |
| | | 181 | ccagccctga agtcccgagc catgatcacc aaggacacct cgaagaatga gatttatctg | |
| | | 241 | acactgaaga gcctgacaag cgaggacacg gccgtctatt actgtgcagg agcctggggc | |
| | | 301 | ggaaattact acgaaaattt ttttattaat ggtgtagaga attggggcca gggtatcctg | |
| | | 361 | gtcaccgtct cg | |
| 3-7 | DQ125439 | 1 | caggtgcagc tgaaggagtc gggacctggc ctggtgaagc cctcgcagac cctctccctc | 29 |
| | | 61 | acctgcactg tctctggatt atctttgagc agtagttgtg tacaatgggt ccgccaggtt | |
| | | 121 | ccaggaaaag ggctggaata cgtcggtagg atagttagta gtggtggtgg tctaacctac | |
| | | 181 | aacccggccc tgaagtcccg agccagcatc accagagaca cttcaaagag ccaggtttat | |
| | | 241 | ctgacgctga acagcctgac agacgaggac acggccgtct attactgtac aggggccctg | |
| | | 301 | aatactcact acagttcata cgcgggttat ggtatagact actggggcca gggtatcctg | |
| | | 361 | gtcaccgtct cg | |
| 3-8 | DQ125440 | 1 | caggtgcagc tgaaggagtc agggcctggc ctggtgaagc ccgcgcagac ccttaccctt | 30 |
| | | 61 | acctgcactg tctctggatt acacttgaac agtgacgcgg tagtgggctg ggtccgtcag | |
| | | 121 | gctccaggaa aggggctgga atttgttggt ggattgtcta atacaggacg tgcaaactac | |
| | | 181 | aatccagccc tgaagtcccg agccatcatc accaaggaca cctcaaagag ccaggtttat | |
| | | 241 | ctgaccctga acagcctgac aagcgaggac acggccgact atttttgtgc aggaggtaga | |
| | | 301 | atgttcgatt atgtttatgg cggctattac gaaatacaat attggggcca gggtatcctg | |
| | | 361 | gtcaccgtct cg | |
| 3-9 | DQ125441 | 1 | caggtgcaac tgaaggagtc gggacctggc ctggtgaagc cctcgcaggc cctgtccctc | 31 |
| | | 61 | acctgtacta tctctgggtt ctctttgacc agtcacggtg taggctgggt ccgccaggct | |
| | | 121 | ccaggaaaag ggctggaatt tgtcggtagt atatggacta cgggacagac aatcaacaat | |
| | | 181 | ccaaccctga agtcccgagt cagcatcact agggacaccg ggctgaacca agtttcactg | |
| | | 241 | acgttgaatg agttgacaag cgaggacacg gccgtctatt attgtgcagg aggcgcgatt | |
| | | 301 | tccgattacg actttttcgg ttttcgtggt atgttcagca tttatgatgt gcagtattgg | |
| | | 361 | ggccagggta tcctggtcac cgtctcg | |
| 3-12 | DQ125442 | 1 | caggtgcagc tgaaggagtc aggacctggc ctggtgaagc cctcgcagac cctctccctc | 32 |
| | | 61 | gtctgcactg tctctggatt cagtttgaac agttggggtg taggctgggt ccgccaggct | |
| | | 121 | ccaggaaaag ggctggagga agttggtgga agtcagattg gtgggaatgc aaactacaat | |
| | | 181 | ccagccctgg agtcccgagc cagcatcacc aaggacgcct caaagagcca agtttatctg | |
| | | 241 | acgctgaaca gcctgacaga agaggacacg gccgtctatt actgtacagg aggttacaac | |
| | | 301 | tggaatcttg ggactaatag ggaccgtata acgtactggg gccagggtat cctggtcacc | |
| | | 361 | gtctcg | |
| 3-13 | DQ125443 | 1 | caggtgcagc tgaaggagtc aggacctggc ctggtgaagc ccgggcagac cctctccctc | 33 |
| | | 61 | tcctgcactg tctctggatt gtcattgagc acaaatactg taggttgggt ccgccaggct | |
| | | 121 | ccaggaaaag gatgggaata tgttgctgcg ttatacgcgg atgcagatgg agattataat | |
| | | 181 | ccagtccttc agtcccgagc cagcatcacg aaggacacct ccaagaacca ggtctttctg | |
| | | 241 | acgctagaca cactgacgag cgaggacacg gccgtctatt actgcacagg gggagtcttc | |
| | | 301 | tccgtccccg tcggtactgg atatacttac tatgaatcgg gaatactata ctggggccag | |
| | | 361 | ggtatcctgg tcaccgtctc g | |
| 3-15 | DQ125444 | 1 | caggtgcaac tgaaggagtc aggacctggc ctggtgaacc cctcgcagac cctgtccctc | 34 |
| | | 61 | acctgctttg tctctggatt ctctttgacg agttggcatg taggctgggt ccgccaggct | |
| | | 121 | ccaggaaaag ggctggaatt tgtcggtggt atacctgtca tcggagaggc atactacaac | |
| | | 181 | ccagtgctga agtcccgaat cagcattact aaggacacct cgaagagcca agtttatctg | |
| | | 241 | acgctgaaca gcctgacaga cgaggacacg gccgtctatg cctgtgcgag gttaaggaac | |
| | | 301 | tggtatggtg attactacag tgacatggac tattggggcc agggtatcct ggtcaccgtc | |
| | | 361 | tcg | |
| 4-1 | DQ125445 | 1 | caggtgcagc tgcaggagtc aggacctggc ctggtgaagc cctcgcagac cctgtccctc | 35 |
| | | 61 | acctgcactg tctctggact ctctttgaac agttacgatg taaactgggt ccgccaggct | |
| | | 121 | ccaggaaaag ggctggaagt agttggtagt ataagtgaca gtggaattgc ggtgtacaac | |
| | | 181 | ccagccctga agtcccgagc cagcatcacc aaggacacct caaacagtca agtttatctg | |
| | | 241 | acgctgaaca gcctgacagg cgaggacacg gccgtctatt actgtgcgag agggaatttt | |
| | | 301 | gcgtttgact actggggcca gggtatcctg gtcaccgtct cg | |
| 4-2 | DQ125446 | 1 | caggtgcaac tgaaggagtc aggacctggc ctggtgaagc cctcgcagac tctctccctc | 36 |
| | | 61 | acctgcgctg tctctggatt ctctttgaga gatgccgcca tgggctgggt ccgccaggct | |
| | | 121 | ccaggaaggg gtctggaata catcggttct atgtatatta gagaagacta caatccagcc | |
| | | 181 | ctgaagtccc gagccagcgt caccaaggac acaaaggaga gccgaagtta tctgacactg | |
| | | 241 | aacgcgctga caagtgagga cacggccgtc tattggtgtg taggggatgt tggcactgga | |
| | | 301 | tactactggg gccagggtat cctggtcacc gtctcg | |
| 4-3 | DQ125447 | 1 | caggtgcagc tgaaggagtc gggacctggc ctggtgaagc cctcgcagac cctctccctc | 37 |
| | | 61 | acctgcactg tctctggatt ctctttgagc accaccggtg tgggctgggt ccgccaggct | |
| | | 121 | ccaggaaaag ggctggaatt tgttggtggt gtacctagta gtggaagtgc aaactacaat | |
| | | 181 | ccagccctga agtcccgatg cagcatcacc aaggacgaat caaagagcca agtttatctg | |
| | | 241 | acgctgaaca gcctgacaag cgaggacacg gccgtctata tatgtgcagg aggcttctac | |
| | | 301 | aatacattgg ataaggggat aaactattgg ggccagggta tcctggtcac cgtctcg | |
| 4-4 | DQ125448 | 1 | caggtgcaac tgaaggagtc aggacctggc ctggtgaagc cctcgcagac cctgtccctc | 38 |
| | | 61 | acctgcacta tctctggatt ctctttgacc agtgccagtg tagactgggt ccgccaggct | |
| | | 121 | ccaggaaaag ggctggaatt tgttggtggt atagcgacta gtgggcgtgc aaattacaac | |
| | | 181 | ccagtcctga agtcccgcgc cactatcacc agagacacct caaagagcca agtttatctg | |
| | | 241 | acgctgaaca gtctgacagg cgaggacacg gccgtctatt actgtgcgga atcctactat | |
| | | 301 | gatggtgttg gtggtaatta ctacttttgg ggccagggta tcctggtcac cgtctcg | |
| 4-5 | DQ125449 | 1 | caggtgcaac tgaaggagtc aggacctggc ctggtgaagc cctcgcagac cctttccctc | 39 |
| | | 61 | acctgcaccg tctctggaat gtctttgagc accaacactg taggctgggt ccgccaggct | |
| | | 121 | ccaggaaaag ggctggaata cgttggtcta atctatggta tgaaaagtgc agagtacaat | |
| | | 181 | ccagccctga agtcccgagc cagtatcacc aaggacacct caaatagtca agttcttctg | |
| | | 241 | acgctgaata gcctgacaag cgaggacacg gccgtctact actgtgcagg gggtgaagcc | |
| | | 301 | tggggtccaa tgtatagttc gaacgaagaa aaaaatggtg tggaatactg gggccagggt | |
| | | 361 | atcctggtca ccgtctcg | |
| 4-6 | DQ125450 | 1 | caggtgcaac tgcaggagtc aggacctggc ctggtgaagc cctcgcagac cctctccctc | 40 |
| | | 61 | acctgcactg tctctggaat ctctttgacc gattacaatg tagactgggt ccgccaggct | |
| | | 121 | ccaggaaaag ggctggaatt tgttggtgga ctatggacta atggacaatc gaactacaat | |
| | | 181 | ccagccctga agtcccgagc cagaatcacc aaggacacct caaagagtca agtttatctg | |
| | | 241 | acgctgaaca gcctgacaag cgaggacacg gccgtctatt actgtgaggg ttatggtaat | |
| | | 301 | tcctggcagc caccgcacta ctggggccag ggtatcctgg tcaccgtctc g | |
| 4-7 | DQ125451 | 1 | caggtgcagc tgaaggagtc aggacctggc ctggtgaagc cctcgcagac cctgtccctc | 41 |
| | | 61 | acctgcactg tctctgggaa cgatttgaga agttttggcg tagcctgggt ccgccaggct | |
| | | 121 | ccaggaaaag gcctggaatt tgttggtggt gtagccaggt ttggcagccc ttactacaac | |
| | | 181 | ccagccctga agtcccgggc catcatcacc aaggacacct caaagaagga aagtgtgctg | |
| | | 241 | acgttaaata gcgtgacagg cgaggacacg gccgtctatt ggtgtgcagg gggatatggt | |
| | | 301 | gatgaatcct ggggaccctg gggccagggt atcctggtca ccgtctcg | |
| 4-8 | DQ125452 | 1 | caggtgcaac tgaaggagtc aggacctggc ctggtgaagc cctcgcagac cctctccctc | 42 |
| | | 61 | acctgcactg tctctgcgtt atctttgagc agtgctggtg tgggctgggt ccgccaggct | |
| | | 121 | ccaggaaaag ggctggaatt tgttgctggt atagttggtg atggtggtac gtacgccaac | |
| | | 181 | ccagccctga ggtcccgagc cagcatcacc aaggacacct caaagagcca agtttatctg | |
| | | 241 | acgctgaaca tgctgacaag cgaggacacg gccgtctatt actgtgcagg aagcttggag | |
| | | 301 | tttagtggct ggggagttat gcgctacggt ataaactact ggggccaggg tatcctggtc | |
| | | 361 | accgtctcg | |
| 4-9 | DQ125453 | 1 | caggtgcaac tgcaggagtc aggacctggc ctggtgaagc cctcgcagac cctctccctc | 43 |
| | | 61 | acctgcactg tctctggatt atctttgagc agtaatgctg taggctgggt ccgccaggct | |
| | | 121 | caaggaaaag ggctggaata tgttgatagt ataggcaaca gtgaaagtgc aaactttaac | |
| | | 181 | ccagccctga agtcccgagc cagcatcacc gaggacacct caaagagccg agtttatctg | |
| | | 241 | acgctgaaca gcctgacaag cgaggacacg gccgtctatt actgtgcagc ccaatatgat | |
| | | 301 | tactttgctg gtgcttatgg cctcatccct tatgctataa agtactgggg ccagggtatc | |
| | | 361 | ctggtcaccg tctcg | |
| 4-10 | DQ125454 | 1 | caggtgcaac tgaaggagtc gggacctggc ctggtgaagc cctcgcagac cctgtccctc | 44 |
| | | 61 | acctgcactg tctctggatt ccctttgagc agttacggtg taggctgggt ccgccaggct | |
| | | 121 | ccaggaaaag ggctggaatc ggttggtgaa atagctagta gtggaagtgc aaactacaac | |
| | | 181 | ccagccctga agtcccgagc cagcatcacc aaggacacct caaagagcca agtttatctg | |
| | | 241 | acgctgaaca gcctgacaag cgaggacacg gccgtctatt attgtacagg atggggactg | |
| | | 301 | agactgtact actggggcca gggtatcctg gtcaccgtct cg | |
| 4-11 | DQ125455 | 1 | caggtgcagc tgcaggagtc aggacctggc ctggtgaagc cctcgcagac cctctccctc | 45 |
| | | 61 | acctgtactg tctctggatt atcactgagc agtaatgtgt taggctgggt ccgccaggct | |
| | | 121 | cccggaaaag ggctggaatg gattggtgga atatatggaa gtgcaagtcc aaactataat | |
| | | 181 | ctaaccctga aggcccgagg cagcatcacc aaggacacct caaagagcca agtgtatctg | |
| | | 241 | acgctaactg ggatgacaga ggaggacacg gccgtctatt actgtgcagg aggggctccc | |
| | | 301 | tataattatg ccggtggtaa cattggaaga atgaagtatt ggggccaggg tatcctggtc | |
| | | 361 | accgtctcg | |
| 4-12 | DQ125456 | 1 | caggtgcagc tgaaggagtc aggacctggc ctggtgaagc cctcgcagac cctctccctc | 46 |
| | | 61 | acctgcactg tctctggatt atctttgagc agttatggtg tgggctgggt ccgccaggct | |
| | | 121 | ccaggaaaag gtctggaatt tgttggcggt atacttagta gtggaagggc aaactacaac | |
| | | 181 | ccagccctga agtcccgagc cagcatcacc agggatacaa caaagaacca agtttatctg | |
| | | 241 | acgctgaaca gcctgacagg cgaggacacg tccgtctatt actgtgcgag atcatttgct | |
| | | 301 | agtggtggtt cttactacga ctatgcgata aacttctggg gccagggtat cctggtcacc | |
| | | 361 | gtctcg | |
| 4-14 | DQ125457 | 1 | caggtgcagc tgaaggagtc aggacctggc ctggtgaagc cctcgcagac cctctccctc | 47 |
| | | 61 | aactgcgctg tctctggatt acctttgaga gatgctgccg taggctgggt ccgccaggct | |
| | | 121 | ccaggaaagg gtctggaata tattggttct atgtataatg aagaagacta caatccagac | |
| | | 181 | ctgaagtccc gagccagcgt caccaaggac acctcaaaga gccgagtcac tctgacgctg | |
| | | 241 | aacagtctga caagtgagga cacggccgtc tattactgtg taggggacgg tggctctgga | |
| | | 301 | tactactggg gccagggtat cctggtcacc gtctcg | |
| 4-15 | DQ125458 | 1 | caggtgcaac tgcaggagtc gggcccagga caggtgaagc cctcacagac cctctccctc | 48 |
| | | 61 | acctgcactg tcactggagg atccatcaca aacaagtatt ctagctggac ctggttacgc | |
| | | 121 | cagcctccag ggaagggcct ggaatttatc ggatacatat attatgatgg tagacgttac | |
| | | 181 | tacaatcctt ccttcaagag ccgcacctcc atctccagag acacctccag gaacgagttc | |
| | | 241 | tccctgcagc tgagctccgt gaccgatgag gacgcggccg tatatttttg tgcaggggat | |
| | | 301 | tatggttatg gcggtgtttg gtactcagat ggtgaaaact actggggcca gggtatcctg | |
| | | 361 | gtcaccgtct cg | |

### Example 6

### Characterization of the Horse V_{H} Sequences

A. Pairwise Identity Matrix of the Plurality of V_{H} Sequences

It has been established that V_{H} sequences within families share nucleotide identity estimates of at least 80%, whereas sequence identity among those belonging to different families is at most 75% (Brodeur et al., Eur. J. Immunol., 14:922-930, 1984, the disclosure of which is incorporated herein by reference).

Out of a total of 1081 pairwise nucleotide comparisons of the horse V_{H} sequences, 54 resulted in identity values above 90%, 620 comparisons generated estimates in the range of 80-89%, 359 from 70-79%, and 48 comparisons resulted in values below 70%. Identity estimates above 90% were mainly generated by comparisons with six horse V_{H} sequences: 1-5, 2-3, 2-14, 3-5, 4-9, and 4-10. Another twenty-seven sequences yielded estimates in the range of 80-89%. Thirteen sequences (clones 1-7, 2-1, 2-7, 2-9, 2-15, 3-6, 3-8, 3-9, 3-13, 3-15, 4-2, 4-7, and U15150) yielded identity estimates from 70-79%, although two of the comparisons with sequence 2-15 resulted in identity estimates below 70%. All comparisons performed with clone 4-15 resulted in values below 68%.

Considering that the horse V_{H} sequences studied in this work are rearranged IGHV genes isolated from a horse hyperimmunized with scorpion venom, it is possible that the six sequences with identity values above 90% originate from the same germline gene and diverged as a result of somatic mutation. The forty sequences with identity values between 89% and 70% may represent more heavily mutated versions of a small number of germline genes, though given the considerable divergence among them it is more likely that they originated from a considerable number of IGHV germline genes.

In the case of clone 2-15, the long insertion in the H2 loop of this clone contributed to the identity values below 70% with respect to two other sequences. Clone 4-15, on the other hand, presented identity values below 70% in all forty-six pairwise comparisons, thus pointing to the fact that this sequence originated from a germline gene belonging to another IGHV gene family.

B. Sequence Analysis and Structural Repertoire of Equine Canonical Structures

The horse V_{H} sequences were analyzed with a combination of programs available on the Internet such as ExPASy (http://www.expasy.org/tools/dna.html),Ident and Sim (http://www.123genomics.com/files/analysis.html).

Canonical structures of the hypervariable loops have been defined elsewhere as follows (Chothia et al., J. Mol. Biol., 196: 901-917, 1987; Chothia et al., Nature, 342: 877-883, 1989; Chothia et al., J. Mol. Biol., 227: 799-817, 1992; Tramontano et al., J. Mol. Biol., 215: 175-182, 1990; Al-Lazikani et al., J. Mol. Biol., 273: 927-948, 1997; and Almagro et al., Mol. Immunol. 34(16-17):1199-214, 1997, the disclosures of which are incorporated herein by reference in their entireties). In structural terms, H1 has been defined as the hypervariable loop beginning at position 26 and finishing at position 32. Three different sizes have been identified for this loop: canonical structures type 1 (7 residues), type 2 (8 residues) and type 3 (9 residues). The pattern of residues compatible with these canonical structure types for H1 have been described elsewhere (Chothia et al., 1987, supra; Chothia et al., 1989, supra; Chothia et al., 1992, supra; Tramontano et al., 1990, supra; Al-Lazikani et al., 1997, supra; and Almagro et al., 1997, supra).

H2 is defined as the hypervariable loop located from position 52 to position 56. So far, five different sizes have been found. Early works assigned canonical structural type 1 to the shortest loop (5 residues), the next length (6 residues) to types 2 and 3 (these types share the same length and thus we will refer to these types as 2/3), and type 4, identified with the longest loop (8 residues). Later, two other sizes for H2 were distinguished in the functional VH gene segments of humans: one having 7 residues (between the size of types 2/3 and type 4) named type 5 and one shorter than type 1 (4 residues) named type 6. The patterns of residues determining the different canonical structures for H2 have been described in detail in previous works (Chothia et al., 1987, supra; Chothia et al., 1989, supra; Chothia et al., 1992, supra; Tramontano et al., 1990, supra; Al-Lazikani et al., 1997, supra; and Almagro et al., 1997, supra).

The canonical structural of the horse V_{H} repertoire was determined. In H1, two out of the three canonical structures known at present are encoded by the most numerous horse gene family, IGVH1. Clone 4-15, which defines the horse IGVH2 gene family, has the third canonical structure described for H1. Since it has been suggested that the structural repertoire is family-specific (Almagro et al., 1997, supra), the difference of canonical structures at H1 in clone 4-15 with respect to the remaining equine sequences provides an additional element to validate this sequence as member of a new horse V_{H} gene family.

In H2, 38 out of the 47 (80%) horse sequences have type 1. Two clones have one residue shorter than type 1 (type 6) and one clone presents an additional amino acid in H2 thus generating either types 2 or 3. A total of six clones have lengths that do not correspond with any of the canonical structures so far described. Clones 1-7, 2-4, 4-2, and 4-14 have shorter loops than type 6, with lengths ranging from 2 to 3 amino acids. Clone 2-7 is even shorter, with a complete deletion of H2. Clone 2-15, on the other hand, has an unusually long H2 loop. By definition, these lengths should generate new canonical structures at H2.

The structural repertoire encoded in the human IGHV germline genes is dominated by type 2 and 3 at H2 (56%). H2 type 1, which is the most abundant canonical structure in horses, is present in 35% of the human sequences. Furthermore, no loop shorter than type 6 or longer than type 4 is found in humans, whereas in horses these loop lengths are found in 13% of the sequences. The structural repertoire of horses thus seems to be shorter than the human genuine gene repertoire and with length variations not seen in human germline genes.

The unusually long insertion at H2 in clone 2-15 consists of the repetitive pattern IGNSGST/IGNSGKT, a characteristic that is believed to be a signature of DNA polymerase stuttering during somatic hypermutation (Wilson et al., J. Exp. Med., 187:59-70, 1998, the disclosure of which is incorporated herein by reference in its entirety). Comparative analyses of human V_{H} germline genes and rearranged sequences indicate that somatic deletions and insertions occur in H2 in members of the human VH2 and VH4 gene families (Wilson et al., 1998, supra; de Wildt et al., J. Mol. Biol., 294:701-710, 1999, the disclosure of which is incorporated herein by reference). These mutational events generate shorter H2 loops that type 6 or longer than type 4. Assuming that these lengths are somatically generated in the horse as well, it is remarkable however that in humans these events occur with a frequency of 2%, whereas in horses the frequency of these events is 13% (6 out of 47). This six-fold increase in horses might be a consequence of the fact that the equine sequences obtained are derived from a hyperimmunized animal, where diversification of the structural repertoire encoded in germline genes has been under positive selection.

C. H3 Length Distribution and Amino Acid Composition

Once the variable gene region of the equine sequences was characterized, the repertoire of H3 loops was analyzed.

H3 length distribution in equine sequences was found to follow a bimodal model, with most of the sequences ranging from 10 to 21 amino acids. This latter group of lengths is normally distributed with an average of 16.9±4 amino acid residues. Previously, Schrenzel et al. (Immunogenetics, 45:386-393, 1997, the disclosure of which is incorporated herein by reference in its entirety) reported that H3 lengths for horse sequences ranged from 12 to 17 amino acids, with half of them having 14 residues.

Horse H3 loops have only two cysteine residues out of 727 amino acids (0.3%) analyzed. The only two cysteine residues in horse H3 loops are found in the same clone, namely 3-2 and are six residues apart from each other. This suggests that they form an intra-chain disulfide bond that constrains the loop structure. In the remaining forty-six horse sequences, the absence of cysteine residues may thus result in less constrained loops. Less constrained H3 loops may be able to search more exhaustively the space of conformations, which creates more structural solutions to recognize diverse antigens.

Horse H3 loops also have a high content of glycine and tyrosine content. The high content of glycine in horses could enhance the loop flexibility and allow bulky amino acids in their immediate vicinity, like tyrosine and phenylalanine. Tyrosine is a very versatile residue in terms of molecular interactions. It could contribute to the antigen-antibody complex with stacking interactions, hydrogen bonds, as well as π and hydrophobic interactions. This way, the overuse of tyrosine in equine H3 loops, together with the potential flexibility conferred by the absence of cysteine and the high content of glycine, could be an important factor in the expansion of the repertoire of antigen-binding sites provided by the observed diversity of canonical structures at H1 and H2.

### Example 7

### Polyclonal ELISA After Panning of the Plurality with a Scorpion Toxin

In order to determine that the plurality of chimeric scFv antibodies are immunospecific for a scorpion toxin, two rounds of selection against cll1, one of the two toxins responsible for the lethality of the C. limpidus limpidus venom, were conducted.

Selections were conducted loosely as previously described (Marks et al., J. Mol. Biol., 222: 581-597, 1991). Briefly, Immunotubes (Nunc Maxisorb; Cat. #12-565-135; Fisher-Scientific) were coated with 4 mL of cll1 (10 μg/mL) in carbonate buffer (Bicarbonate 50 mM NaHCO₃ 50 mM, pH: 9.6). The coating solution was incubated for one hour at 37° C., discarded, and the immunotubes were blocked with MPBS (PBS+2% skim powder milk; Publix Brand) for one additional hour at 37° C. For the first round of selection, the plurality (10¹³ phages) was diluted in 4 mL of MPBS and added to the cll1-coated Immunotubes, incubated for one hour at room temperature with rotation and then left to stand for an additional hour at room temperature.

Unbound phages were washed away by 10 washes with TPBS (PBS+0.1% Tween 20) and 10 additional washes with PBS. Bound phages were eluted by trypsinization (Goletz et al., J Mol Biol. 315(5):1087-97, 2002) and used to infect exponentially growing TG1 cells (OD₆₀₀=0.4). Infected cells were grown overnight at 37° C. in 2×TY-agar plates containing 100 μg/mL carbenicillin and 1% glucose. Cells infected with the selected phages were scraped from the plate and phage particles were rescued with the KM13 helper phage as described above in the Example 4. The second round of selection was conducted following the same procedure as the first round of selection, but using the phage (10¹³ phage) eluted from the first round.

Enrichment of the plurality of chimeric scFv antibodies immunospecific for cll1 was assessed by ELISA. Nunx Maxisorp ELISA plates were coated with 50 μL of cll1 5 μg/mL in carbonate buffer for one hour at 37° C. and blocked with MPBS for an additional hour at 37° C. 50 μL of the polyclonal PEG-purified phage after the first and the second rounds of selections were incubated in the cll1-coated plates for one hour at room temperature with shaking. The plates were washed with 0.1% TPBS (PBS containing 0.1% Tween 20). Anti-M13: horseradish peroxidase (HRP) conjugated (Amersham-Biotech) in a 1:5000 dilution in BSA-PBS was added to the wells and incubated for one hour. Plates were washed and 50 μL per well of TMB solution (Promega) was added. The reaction was stopped after ten minutes with HCl 1N and the absorbance read at 450 nm in a microplate reader.

The results of the first round of selection detected non-specific binding of the plurality of chimeric scFv antibodies to cll1 (baseline). The results of the second round of selection indicated an increase in cll1 binding with an EC₅₀ (titer at 50% of the ELISA signal) of 1×10¹¹ cfu/mL. This result indicates a considerable increase in the population of chimeric scFv antibodies that recognize cll1.

In order to confirm that the plurality of chimeric scFv antibodies were immunospecific for cll1, another ELISA was performed following the procedure described above but using HEL (Hen Egg White Lysozyme) instead of cll1 to coat the ELISA plates. The EC₅₀ for HEL was 1×10¹² cfu/mL, which indicates that only 10% of the phage recognizes HEL, or that the scFv-phage recognizes HEL with an affinity one order of magnitude below the one used to recognize cll1, or a combination of both. These results indicate that the plurality of chimeric scFv antibodies isolated after the second round of panning with cll1 was immunospecific for cll1.

1. A plurality of chimeric single chain Fv (scFv) antibodies comprising at least two or more chimeric scFv antibodies that are immunospecific for different/distinct epitopes, said chimeric scFv antibodies individually comprising a horse Fv domain and a non-horse Fv domain.

2. The plurality of chimeric scFv antibodies of claim 1, wherein the antibodies in the plurality are biased toward immunospecific recognition of toxin epitopes.

3. The plurality of chimeric scFv antibodies of claim 2, wherein the toxin is a neurotoxin.

4. The plurality of chimeric scFv antibodies of claim 1, wherein each of said horse Fv domain is a VH fragment and each of said human Fv domain is a VL fragment.

5. The plurality of chimeric scFv antibodies of claim 4 wherein each human Fv domain in the plurality is identical.

6. The plurality of chimeric scFv antibodies of claim 4 wherein each human Fv domain in the plurality is VL fragment A27/Jk1 (SEQ ID NO: 2).

7. The plurality of chimeric scFv antibodies of claim 1, wherein each of said horse Fv domain is a VL fragment and each of said human Fv domain is a VH fragment.

8. The plurality of chimeric scFv antibodies of claim 7 wherein each human Fv domain in the plurality is identical.

9. The plurality of chimeric scFv antibodies of claim 4, wherein the VH fragment is selected from a phage library.

10. The plurality of chimeric scFv antibodies of claim 4, wherein the VH fragment comprises one or more fragments selected from the group consisting of an H1 fragment, an H2 fragment, and an H3 fragment.

11. The plurality of chimeric scFv antibodies of claim 10, wherein the VH fragment is an H1 fragment.

12. The plurality of chimeric scFv antibodies of claim 10, wherein the VH fragment is an H2 fragment.

13. The plurality of chimeric scFv antibodies of claim 10, wherein the VH fragment is an H3 fragment.

14. The plurality of chimeric scFv antibodies of claim 10, wherein the VH fragment comprises an H1 fragment and an H2 fragment.

15. The plurality of chimeric scFv antibodies of claim 10, wherein the VH fragment comprises an H1 fragment and an H3 fragment.

16. The plurality of chimeric scFv antibodies of claim 10, wherein the VH fragment comprises an H2 fragment and an H3 fragment.

17. The plurality of chimeric scFv antibodies of claim 8, wherein the VH 10 fragment comprises an H1 fragment, an H2 fragment, and an H3 fragment.

18. The plurality of chimeric scFv antibodies of claim 4, wherein the VL fragment comprises one or more fragments selected from the group consisting of an L1 fragment, an L2 fragment and an L3 fragment.

19. The plurality of chimeric scFv antibodies of claim 18, wherein the VL fragment is an L1 fragment.

20. The plurality of chimeric scFv antibodies of claim 18, wherein the VL fragment is an L2 fragment.

21. The plurality of chimeric scFv antibodies of claim 18, wherein the VL fragment is an L3 fragment.

22. The plurality of chimeric scFv antibodies of claim 18, wherein the VL fragment comprises an L1 fragment and an L2 fragment.

23. The plurality of chimeric scFv antibodies of claim 18, wherein the VL fragment comprises an L1 fragment and an L3 fragment.

24. The plurality of chimeric scFv antibodies of claim 18, wherein the VL fragment comprises an L2 fragment and an L3 fragment.

25. The plurality of chimeric scFv antibodies of claim 18, wherein the VL fragment comprises an L1 fragment, an L2 fragment and an L3 fragment.

26. The plurality of chimeric scFv antibodies of claim 1, wherein said chimeric scFv antibodies comprise one or more natural or non-natural modifications which do not eradicate the affinity of said chimeric scFv antibodies to an epitope.

27. The plurality of chimeric scFv antibodies of claim 1, wherein said chimeric scFv antibodies comprise one or more natural or non-natural modifications which do not substantially alter the affinity of said chimeric scFv antibodies to an epitope.

28. The plurality of chimeric scFv antibodies of claim 26, wherein the modification(s) is selected from the group consisting of deletion, insertion, and substitution.

29. The plurality of chimeric scFv antibodies of claim 1 wherein said chimeric scFv antibodies are conjugated to a polypeptide.

30. The plurality of chimeric scFV antibodies of claim 29 wherein the polypeptide is a fragment of a second antibody.

31. The plurality of chimeric scFv antibodies of claim 1 wherein said chimeric scFv antibodies are conjugated to a water soluble polymer.

32. The plurality of chimeric scFv antibodies of claim 31 wherein said water soluble polymer is polyethylene glycol.

33. The plurality of chimeric scFv antibodies of claim 1, wherein said chimeric scFv antibodies are labeled.

34. The plurality of chimeric scFv antibodies of claim 33, wherein said label is selected from the group consisting of enzymes, radioisotopes and fluorescent compounds.

35. A method of mutagenesis of the plurality of chimeric scFv antibodies according to claim 1, the method comprising:
a) mutagenizing genes encoding the individual chimeric scFv antibodies; and
b) expressing the genes to produce mutagenized chimeric scFv antibodies.

36. The method of claim 35 further comprising the step of screening said mutagenized chimeric scFv antibodies to select for a desired structure or function.

37. The method of claim 35, wherein the mutagenizing is accomplished by site-directed mutagenesis.
